(19) United States
(12) Patent Application Publication
(10) Pub. No.: US 2015/0044193 A1
(43) Pub. Date: Feb. 12, 2015

(54) COMPOSITIONS AND METHODS FOR THE TREATMENT OF ALTERED a-SYNUCLEIN FUNCTION

(71) Applicants: Neuraltus Pharmaceuticals, Inc., Palo Alto, CA (US); Parkinson's Institute, Sunnyvale, CA (US); The Hospital for Sick Children, Toronto (CA)

(72) Inventors: Amy B. Manning-Bog, Fremont, CA (US); Birgitt Schüle, Menlo Park, CA (US); J. William Langston, Los Altos Hills, CA (US); Clifford A. Lingwood, Toronto (CA); Michael S. McGrath, Burlingame, CA (US); Arasteh Ari Azhir, Los Altos, CA (US)

(21) Appl. No.: 14/305,959

(22) Filed: Jun. 16, 2014

Related U.S. Application Data
(63) Continuation of application No. 13/128,222, filed on Nov. 11, 2011, now abandoned.
(60) Provisional application No. 61/199,243, filed on Nov. 14, 2008.

(51) Int. Cl. A61K 31/336; A61K 31/4164; A61K 38/47

(52) U.S. Cl. CPC A61K 31/336; A61K 38/47; A61K 31/4164; C12Y 302/01045
USPC 424/94.61; 514/400; 514/475

(57) ABSTRACT

Compositions and methods are provided for treating a disorder characterized by α-synuclein dysfunction and/or altered lipid metabolism.

### CROSS-REFERENCE

This application is a continuation of U.S. patent application Ser. No. 13/128,222, filed Nov. 11, 2011, which is a 371 National Stage application of PCT Application No. PCT/US09/56116, filed Sep. 4, 2009, which claims the benefit of U.S. Provisional Application No. 61/199,243, filed Nov. 14, 2008, the contents of each application are incorporated herein by reference.

### BACKGROUND OF THE INVENTION

Clinical and neuropathological links have been reported between α-synucleinopathies and lipid metabolism diseases, for example between Parkinson's disease (PD) and non-neuronopathic (type 1) Gaucher disease. α-synuclein is dysregulated in Parkinson's Disease and several other neuronal diseases, commonly referred to as α-synucleinopathies. Higher than normal expression levels of α-synuclein have been shown to cause neurodegeneration in humans (Singleton et al., 2003, Chartier-Harlin et al., 2004, Farrer et al., 2004, Fuchs et al., 2007), and changes in α-synuclein levels are associated with toxicity in in vitro and in vivo PD models (Manning-Bog et al. 2002; Vila et al. 2001; Sherer et al. 2003). Thus, depending on cellular conditions, α-synuclein alterations may be a risk factor for neuronal dysfunction and even degeneration. Gaucher disease (GD) is caused by a deficiency of glucocerebrosidase (GCase) which, under normal conditions, hydrolyzes glucocerebroside (GC) to glucose and ceramide (Butters, 2007, Choy et al., 2007, Guggenbuhl et al., 2008, Hruska et al., 2008).

Clinical reports have suggested an association of type 1 Gaucher disease with a form of early onset Parkinson's disease (PD) that is often poorly responsive to levodopa (Neudorfer et al., 1996, Machaczka et al., 1999, Tayebi et al., 2001, Varkonyi et al., 2002, Bembi et al., 2003). Subsequently, a number of genetic screens of patients diagnosed with PD and PD-like diseases (collectively referred to as parkinsonism) for sequence variants in the gene that encodes for GCase (GBA) have also supported this association (Tayebi et al., 2003, Goker-Alpan et al., 2004, Lwin et al., 2004, Aharon-Peretz et al., 2005, Clark et al., 2005, Sato et al., 2005, Sidransky, 2006, Kono et al., 2007). These studies suggest that certain GBA mutations could be genetic risk factors for PD, and raise the possibility that even small variations or heterozygous changes in the gene for GCase may enhance neuronal vulnerability to degenerative changes.

Further evidence for an association between PD and Gaucher disease comes from a neuropathological study of Gaucher disease subjects with homozygous GBA mutations. This study reported four patients who were diagnosed with the type 1 Gaucher disease (with genetic confirmation), parkinsonism and dementia. A correlation was revealed between the pattern of inclusion body deposition (α-synuclein pathology—Lewy body-like inclusions) and abnormal GCase immunoreactivity.

Finally, experimental studies support the idea that α-synuclein may provide a biological link between Gaucher disease and parkinsonism. Defects in GC degradation result in the accumulation of glycolipids within lysosomes, an intracellular site for protein clearance as well as lipid catabolism. Within the cell, α-synuclein metabolism occurs, at least in part, via the lysosomal clearance pathway (Gosavi et al., 2002, Lee et al., 2004, Ravikumar et al., 2005, Lee et al., 2008), and within the lysosome, α-synuclein binds to lipid-containing species including glycosphingolipids (Schlossmacher et al., 2005) and lipofuscin, an observation made in both PD brain (Braak et al., 2001) and mouse models of the disease (Meredith et al., 2002). There is a need for therapeutic agents and therapeutic methods to treat conditions and diseases associated with altered α-synuclein, lysosomal storage and clearance, and lipid metabolism. The present invention provides methods and compositions and methods that satisfies these needs.

### SUMMARY OF THE INVENTION

The present invention describes methods of modulating α-synuclein function, lipid metabolism and lysosomal storage by using agents that modulate α-synuclein function, lysosomal storage and lipid metabolism, in particular glycosphingolipid metabolism.

The present invention describes methods of modulating α-synuclein and lipid metabolism for the treatment of disease.

In one aspect, the invention provides a method of treating a condition characterized by α-synuclein dysfunction by administering an agent that alters lipid metabolism. In one embodiment the condition is selected from Parkinson's disease, Parkinson's disease with accompanying dementia, Lewy body dementia, Lewy body variant of Alzheimer's disease, Huntington's disease, Alzheimer's disease with Parkinsonism, and multiple system atrophy. In one embodiment the α-synuclein dysfunction is in astrocytes. In another embodiment α-synuclein dysfunction is characterized by a dysfunction in α-synuclein fibrillation, ubiquitination, trafficking, subcellular compartmentalization, synaptic targeting, lysosomal storage, or lipid-interactions. In another embodiment lipid metabolism is altered by decreasing ceramide levels with the use of MDR inhibitors. In related embodiments lipid metabolism is altered by decreasing a buildup of at least one glycosphingolipid or by altering glycosphingolipid metabolism. In a specific embodiment the glycosphingolipid is glucocerebroside. In a related embodiment the agent that alters lipid metabolism is selected from MDR inhibitors, glucocerebrosidases, and HMG-CoA reductase inhibitors. In one embodiment the HMG-CoA reductase inhibitor is a statin. In another embodiment the agent is a MDR inhibitor and the MDR inhibitor is chosen from the imidazole derivatives and compounds of Formula 1a, 1b, or 2 having the following formulas depicted immediately below, in the form of a free compound or as its pharmaceutically-acceptable pro-drug, metabolite, analogue, derivative, solvate or salt.

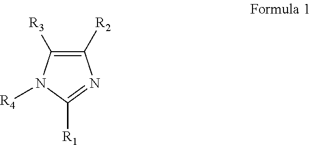

Formula 1 in the form of a free compound or its pharmaceutically acceptable pro-drug, metabolite, analogue, derivative, solvate or salt wherein the substituents R₁, R₂, R₃, and R₄ are defined as described in (a) and (b) below:

(a) when R₁ is selected from the group consisting of:

(i) substituted C₁₋₁₁ alkyl or substituted C₂₋₁₁ alkenyl, wherein the substituents are selected from the group consisting of hydroxy, C₁₋₆ alkyloxy; or

(ii) mono-, di-, and tri-substituted aryl-C₀₋₁₁ alkyl wherein aryl is selected from the group consisting of phenyl, furyl, thienyl wherein the substituents are selected from the group consisting of:
(a) phenyl, trans-2-phenylethenyl, 2-phenylethynyl, 2-phenylethyl, wherein the said phenyl group is mono- or disubstituted with a member selected from the group consisting of hydroxy, halo, C₁₋₄ alkyl and C₁₋₄ alkyloxy,
   (b) substituted C₁₋₆ alkyl, substituted C₂₋₆ alkyloxy, substituted C₂₋₆ alkylthio, substituted C₂₋₆ alkoxycarbonyl, wherein the substituents are selected from the group consisting of C₁₋₆ alkoxy, and C₁₋₆ alkylthio; and
(c) C₁₋₁₁ CO₂R₅, C₁₋₁₁CONHR₅, trans-CH═CHCO₂R₅, or trans-CH═CHCONHR₅ wherein R₅ is C₁₋₁₁ alkyl, or phenyl C₁₋₁₁ alkyl, C₁₋₆ alkoxycarbonylmethyleneoxy;

then R₂ and R₃ are each independently selected from the group consisting of mono-, di, and tri-substituted phenyl wherein the substituents are independently selected from:
(i) substituted C₁₋₆ alkyl,
(ii) substituted C₁₋₆ alkyloxy, C₃₋₆ alkenyloxy, substituted C₃₋₆ alkenyloxy,
(iii) substituted C₁₋₆ alkyl-amino, di(substituted C₁₋₆ alkyl)amino,
(iv) C₃₋₆ alkenyl-amino, di(C₃₋₆ alkenyl)amino, substituted C₃₋₆ alkenyl-amino, di(substituted C₃₋₆ alkenyl)amino,
(v) pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, 4-N—(C₁₋₆ alkylamino C₃₋₆ alkenyl)piperazino,
wherein the substituents are selected from the group consisting of:
   (a) hydroxy, C₁₋₆ alkylalkoxy, C₁₋₆ alkylamino
   (b) C₃₋₆ alkenyloxy, C₃₋₆ alkenylamino, or
   (c) pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, 4-N—(C₁₋₆ alkylamino C₃₋₆ alkenyl)piperazino,

or R₂ and R₃ taken together forming an aryl group or substituted aryl, wherein the substituents are defined as above in (i)-(v);

and R₄ is selected from the group consisting of:
(i) hydrogen;
(ii) substituted C₁₋₁₁ alkyl or C₂₋₁₁ alkenyl wherein the substituents are independently selected from the group consisting of hydrogen, hydroxy, C₁₋₆ alkyloxy, C₁₋₆alkylthio, C₁₋₆ alkylamino, phenyl-C₁₋₆ alkylamino, C₁₋₆ alkoxycarbonyl; or
(iii) substituted aryl C₀₋₁₁ alkyl wherein the aryl group is selected from phenyl, imidazolyl, furyl, thienyl in which the substituents are selected from A(a-c); or

(b) when R₁ is selected from the group consisting of:

Mono-, di-, and tri-substituted aryl-C₀₋₆ alkyl wherein aryl is selected from the group consisting of phenyl, thienyl, and the substituents are selected from the group consisting of:
(a) trans-2-substituted benzimidazolylethenyl, trans-2-substituted benzoxazolylethenyl, trans-2-substituted benzthiazolylethenyl, in which the substituents are selected from the group consisting of hydrogen, hydroxy, halo, trihalomethyl, C₁₋₄ alkyl and C₁₋₄ alkyloxy, C₁₋₄ alkyloxycarbonyl, C₁₋₄ alkylamino, alkyl)amino, C₃₋₆ alkenylamino, di(C₃₋₆ alkenyl)amino, C₁₋₄ alkyloxy-C₁₄ alkylamino, substituted C₁₋₄ alkyl and C₁₋₄ alkyloxy, substituted C₁₋₄ alkyloxycarbonyl, substituted C₁₋₄ alkylamino, di(substituted C₁₋₄ alkyl)amino, substituted C₃₋₆ alkenylamino, di(substituted C₃₋₆ alkenyl)amino, wherein the substituents are as defined above,
(b) trans-2-cyano ethenyl, trans-2-alkylsulfonyl ethenyl, trans-2-alkenylsulfonyl ethenyl, trans-2-substituted alkylsulfonyl ethenyl, trans-2-substituted alkenylsulfonyl ethenyl, in which the substituents are defined above,
(c) C₁₋₆ CO₂R₅, trans-CH═CHCO₂R₅, C₁₋₆CONHR₅, or trans-CH═CHCONHR₅, wherein R₅ is C₁₋₆ alkoxy C₂₋₆ alkyl, amino C₂₋₆ alkyl, C₁₋₆ alkylamino C₂₋₆ alkyl, di(C₁₋₆ alkyl)amino C₂₋₆ alkyl, C₁₋₆ alkylthio C₂₋₆ alkyl, substituted C₁₋₆ alkoxy C₂₋₆ alkyl, substituted C₁₋₆ alkylamino C₂₋₆ alkyl, di(substituted C₁₋₆ alkyl)amino C₂₋₆ alkyl, substituted C₁₋₆ alkylthio C₂₋₆ alkyl, in which the substituents are selected from the group consisting of pyrrolidino, piperidino morpholino, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, 4-N—(C₁₋₆ alkylamino C₃₋₆ alkenyl)piperazino, imidazolyl, oxazolyl, thiazolyl,
(d) C₁₋₆CONR₆R₇, or trans-CH═CHCONR₆R₇, wherein R₆ and R₇ are independently selected from the group consisting of C₁₋₆ alkyl, phenyl C₁₋₆ alkyl, C₁₋₆ alkoxycarbonylmethyleneoxy, hydroxy C₂₋₆ alkyl, C₁₋₆ alkyloxy C₂₋₆ alkyl, amino C₂₋₆ alkyl, C₁₋₆ alkylamino C₂₋₆ alkyl, di(C₁₋₆ alkyl)amino C₂₋₆ alkyl, C₁₋₆ alkylthio C₂₋₆ alkyl, substituted C₁₋₆ alkoxy C₂₋₆ alkyl, substituted C₁₋₆ alkylamino C₂₋₆ alkyl, di(substituted C₁₋₆ alkyl)amino C₂₋₆ alkyl, substituted C₁₋₆ alkylthio C₂₋₆ alkyl, wherein the substituents are selected from the group consisting of pyrrolidino, piperidino, morpholino, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, 4-N—(C₁₋₆ alkylamino C₃₋₆ alkenyl)piperazino, imidazolyl, oxazolyl, thiazolyl,
(e) R₇ C(O) C₁₋₆ alkyl, R₇ C(O) C₂₋₆ alkenyl, in which R₇ is defined as above [2(d)],
(f) HO—C₁₋₆ alkyl-C₂₋₆ alkenyl, R₇—O—C₁₋₆ alkyl-C₂₋₆ alkenyl, R₇NH—C₁₋₆ alkyl-C₂₋₆ alkenyl, R₆R₇N—C₁₋₆ alkyl-C₂₋₆ alkenyl, R₇NH—C(O)—O—C₁₋₆ alkyl-C₂₋₆ alkenyl,

R₆R₇N—C(O)—O—C₁₋₆ alkyl-C₂₋₆ alkenyl, R₇O—C(O)—O—C₁₋₆ alkyl-C₂₋₆ alkenyl, R₇—C(O)—O—C₁₋₆ alkyl-C₂₋₆ alkenyl, wherein R₆ and R₇ is defined as above [2(d)],
(g) R₇—O—C₀₋₃ alkyl-C₃₋₆ cycloalkan-1-yl, R₇NH— C₀₋₃ alkyl- C₃₋₆ cycloalkan-1-yl, R₆R₇N—C₀₋₃ alkyl- C₃₋₆ cycloalkan-1-yl, R₇NH—C(O)—O— C₀₋₃ C₃₋₆ cycloalkan-1-yl, R₆R₇N—C(O)—O— C₀₋₃ alkyl- C₃₋₆ cycloalkan-1-yl, R₇O— C(O)—O— C₀₋₃ alkyl- C₃₋₆ cycloalkan-1-yl, R₇—C(O)—O— C₀₋₃ alkyl- C₃₋₆ cycloalkan-1-yl, R₇O—C(O)—C₀₋₃ alkyl- C₃₋₆ cycloalkan-1-yl, wherein R₇ and is defined as above [B(d)];

then R₂ and R₃ are each independently selected from the group consisting of:
(1) hydrogen, halo, trihalomethyl, C₁₋₆ alkyl, substituted C₁₋₆ alkyl, C₂₋₆ alkenyl, substituted C₁₋₆ alkenyl, C₁₋₆ alkyloxy, substituted C₁₋₆ alkyloxy, C₃₋₆ alkenyloxy, substituted C₃₋₆ alkenyloxy, C₁₋₆ alkylamino, substituted C₁₋₆ alkylamino, C₃₋₆ alkenylamino, substituted C₃₋₆ alkenylamino,
(2) mono-, di-, and tri-substituted phenyl wherein the substituents are independently selected from:
   (i) halo, trifluoromethyl, substituted C₁₋₆ alkyl,
   (ii) C₁₋₆ alkyloxy, substituted C₁₋₆ alkyloxy, C₃₋₆ alkenyloxy, substituted C₃₋₆ alkenyloxy,
   (iii) C₁₋₆ alkyl-amino, di(C₁₋₆alkyl)amino, substituted C₁₋₆ alkyl-amino, di(substituted C₁₋₆ alkyl)amino, C₃₋₆ alkenyl-amino, di(C₃₋₆alkenyl)amino, substituted C₃₋₆ alkenyl-amino, di(substituted C₃₋₆ alkenyl)amino, or
   (iv) pyrrolidino, piperidino, morpholino, imidazolyl, substituted imidazolyl, piperazino, 4-N— C₁₋₆ alkylpiperazino, 4-N—C₃₋₆alkenylpiperazino, 4-N—(C₁₋₆alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, 4-N—(C₁₋₆ alkylamino C₃₋₆ alkenyl)piperazino,
      wherein the substituents are selected from the group consisting of:
   (a) hydrogen, hydroxy, halo, trifluoromethyl,
   (b) C₁₋₆ alkylalkoxy, C₁₋₆ alkylamino, C₁₋₆ alkylthio,
   (c) C₃₋₆ alkenyloxy, C₃₋₆ alkenylamino, C₃₋₆ alkenylthio, or
   (d) pyrrolidino, piperidino, morpholino, imidazolyl, substituted imidazolyl, piperazino, 4-N— C₁₋₆ alkylpiperazino, 4-N—C₃₋₆alkenylpiperazino, 4-N—(C₁₋₆alkoxy C₁₋₆ alkyl)piperazino, 4-N—(C₁₋₆alkoxy C₃₋₆ alkenyl)piperazino, 4-N—(C₁₋₆alkylamino C₁₋₆ alkyl)piperazino, 4-N—(C₁₋₆alkylamino C₃₋₆ alkenyl)piperazino;

with the proviso that at least one of R₂ and R₃ group be selected from [B (2)] and the phenyl and the substituents be selected from (ii)-(v) above; or R₂ and R₃ taken together forming an aryl group such as phenyl, pyridyl, in which the aryl may be optionally substituted, wherein the substituents are defined as above in (i)-(iv);

and R₄ is selected from the group consisting of:
(a) hydrogen;
(b) substituted C₁₋₁₁ alkyl or C₂₋₁₁ alkenyl wherein the substituents are independently selected from the group consisting of:
   (i) hydrogen, hydroxy, C₁₋₆ alkyloxy, C₁₋₆alkylthio, C₁₋₆ alkylamino, phenyl-C₁₋₆ alkylamino, C₁₋₆ alkoxycarbonyl;
   (ii) substituted C₁₋₆ alkyloxy, C₃₋₆ alkenyloxy, substituted C₃₋₆ alkenyloxy,
   (iii) di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, di(substituted C₃₋₆ alkenyl)amino; and
   (iv) pyrrolidino, piperidino, morpholino, imidazolyl, substituted imidazolyl, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, and 4-N—(C₁₋₆ alkylamino C₃₋₆ alkenyl)piperazino; and
(a) aryl C₀₋₁₁ alkyl wherein the aryl group is selected from phenyl, imidazolyl, furyl, thienyl.

In some embodiments, the invention provides a compound of Formula 1a, in the form of a free compound or its pharmaceutically acceptable pro-drug, metabolite, analogue, derivative, solvate or salt, for use in the methods of the invention, wherein:

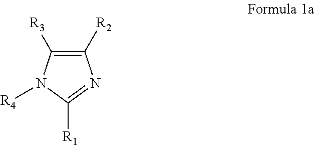

wherein the substituents R₁, R₂, R₃, and R₄ are defined as in A or B:
(A) R₁ is selected from the group consisting of:
(i) substituted C₁₋₁₁ alkyl or substituted C₂₋₁₁ alkenyl, wherein the substituents are selected from the group consisting of hydroxy and C₁₋₆ alkyloxy; and
(ii) mono-, di-, or tri-substituted aryl-O₀₋₁₁ alkyl wherein aryl is selected from the group consisting of phenyl, furyl, and thienyl wherein the substituents are selected from the group consisting of:
   (a) phenyl, trans-2-phenylethenyl, 2-phenylethynyl, or 2-phenylethyl, wherein the phenyl group is mono- or disubstituted wherein the substituents are selected from the group consisting of hydroxy, halo, C₁₋₄ alkyl and C₁₋₄ alkyloxy;
   (b) substituted C₁₋₆ alkyl, substituted C₂₋₆ alkyloxy, substituted C₂₋₆ alkylthio, or substituted C₂₋₆ alkoxycarbonyl, wherein the substituents are selected from the group consisting of C₁₋₆ alkoxy, and C₁₋₆ alkylthio; and
   (c) C₁₋₁₁ CO₂R₅, C₁₋₁₁CONHR₅, trans-CH═CHCO₂R₅, or trans-CH═CHCONHR₅ wherein R₅ is C₁₋₁₁ alkyl, phenyl C₁₋₁₁ alkyl, or C₁₋₆ alkoxycarbonylmethyleneoxy;

R₂ and R₃ are each independently selected from the group consisting of mono-, di, and tri-substituted phenyl wherein the substituents are independently selected from:
(i) substituted C₁₋₆ alkyl;
(ii) substituted C₁₋₆ alkyloxy, C₃₋₆ alkenyloxy, or substituted C₃₋₆ alkenyloxy;
(iii) substituted C₁₋₆ alkyl-amino, di(substituted C₁₋₆ alkyl)amino;
(iv) C₃₋₆ alkenyl-amino, di(C₃₋₆ alkenyl)amino, substituted C₃₋₆ alkenyl-amino, or di(substituted C₃₋₆ alkenyl)amino; and
(v) pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy-C₁₋₆ alkyl)piperazino, 4-N—(C₁₋₆ alkoxy-C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino;

wherein the substituents for (i), (ii), (iii), and (iv) are selected from the group consisting of:
(a) hydroxy, C₁₋₆ alkoxy, or C₁₋₆ alkylamino;
(b) C₃₋₆ alkenyloxy, or C₃₋₆ alkenylamino; and
(c) pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino;

or R₂ and R₃ are taken together to form an aryl group or substituted aryl, wherein the substituents are defined as above in (i)-(iv);

and R₄ is selected from the group consisting of:
(i) hydrogen;
(ii) substituted C₁₋₁₁ alkyl or C₂₋₁₁ alkenyl wherein the substituents are independently selected from the group consisting of hydrogen, hydroxy, C₁₋₆ alkyloxy, C₁₋₆alkylthio, C₁₋₆ alkylamino, phenyl-C₁₋₆ alkylamino, and C₁₋₆ alkoxycarbonyl; and substituted aryl C₀₋₁₁ alkyl wherein the aryl group is selected from phenyl, imidazolyl, furyl, and thienyl in which the substituents are selected from the group consisting of:
   (a) hydroxy, C₁₋₆ alkoxy, or C₁₋₆ alkylamino;
   (b) C₃₋₆ alkenyloxy, or C₃₋₆ alkenylamino; and
   (c)pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino; or

(B) R₁ is selected from the group consisting of:
mono-, di-, and tri-substituted aryl-C₀₋₆ alkyl wherein aryl is selected from the group consisting of phenyl and thienyl, and the substituents are selected from the group consisting of:
(i) trans-2-substituted benzimidazolylethenyl, trans-2-substituted benzoxazolylethenyl, or trans-2-substituted benzthiazolylethenyl, in which the substituents are selected from the group consisting of hydrogen, hydroxy, halo, trihalomethyl, C₁₋₄ alkyl, C₁₋₄ alkyloxy, C₁₋₄ alkyloxycarbonyl, C₁₋₄ alkylamino, di(C₁₋₄ alkyl)amino, C₃₋₆ alkenylamino, di(C₃₋₆ alkenyl)amino, C₁₋₄ alkyloxy-C₁₄ alkylamino, substituted C₁₋₄ alkyl, substituted C₁₋₄ alkyloxy, substituted C₁₋₄ alkyloxycarbonyl, substituted C₁₋₄ alkylamino, di(substituted C₁₋₄ alkyl)amino, substituted C₃₋₆ alkenylamino, and di(substituted C₃₋₆ alkenyl)amino, wherein the substituents are selected from the group consisting of:
   (a) hydroxy, C₁₋₆ alkoxy, or C₁₋₆ alkylamino;
   (b) C₃₋₆ alkenyloxy, or C₃₋₆ alkenylamino; and
   (c)pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino;
(ii) trans-2-cyano ethenyl, trans-2-alkylsulfonyl ethenyl, trans-2-alkenylsulfonyl ethenyl, trans-2-substituted alkylsulfonyl ethenyl, and trans-2-substituted alkenylsulfonyl ethenyl, wherein the substituents are selected from the group consisting of:
   (a) hydroxy, C₁₋₆ alkoxy, or C₁₋₆ alkylamino;
   (b) C₃₋₆ alkenyloxy, or C₃₋₆ alkenylamino; and
   (c)pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino; (iii) C₁₋₆ CO₂R₅, trans-CH═CHCO₂R₅, C₁₋₆CONHR₅, or trans-CH═CHCONHR₅, wherein R₅ is C₁₋₆ alkoxy-C₂₋₆ alkyl, amino-C₂₋₆ alkyl, C₁₋₆ alkylamino-C₂₋₆ alkyl, di(C₁₋₆ alkyl)amino-C₂₋₆ alkyl, C₁₋₆ alkylthio-C₂₋₆ alkyl, substituted C₁₋₆ alkoxy-C₂₋₆ alkyl, substituted C₁₋₆ alkylamino-C₂₋₆ alkyl, di(substituted C₁₋₆ alkyl)amino-C₂₋₆ alkyl, or substituted C₁₋₆ alkylthio-C₂₋₆ alkyl, in which the substituents are selected from the group consisting of pyrrolidino, piperidino morpholino, piperazino, 4-N—C₁₋₆ alkylpiperazino, 4-N—C₃₋₆ alkenylpiperazino, 4-N—(C₁₋₆ alkoxy-C₁₋₆ alkyl)piperazino, 4-N—(C₁₋₆ alkoxy-C₃₋₆ alkenyl)piperazino, 4-N—(C₁₋₆ alkylamino-C₁₋₆ alkyl)piperazino, 4-N—(C₁₋₆ alkylamino C₃₋₆ alkenyl)piperazino, imidazolyl, oxazolyl, and thiazolyl;
(iv) C₁₋₆CONHR₅, or trans-CH═CHCONR₆R₇, wherein R₆ and R₇ are independently selected from the group consisting of C₁₋₆ alkyl, phenyl-C₁₋₆ alkyl, C₁₋₆ alkoxycarbonylmethyleneoxy, hydroxy-C₂₋₆ alkyl, C₁₋₆ alkyloxy-C₂₋₆ alkyl, amino-C₂₋₆ alkyl, C₁₋₆ alkylamino-C₂₋₆ alkyl, di(C₁₋₆ alkyl)amino-C₂₋₆ alkyl, C₁₋₆ alkylthio-C₂₋₆ alkyl, substituted C₁₋₆ alkoxy-C₂₋₆ alkyl, substituted C₁₋₆ alkylamino-C₂₋₆ alkyl, di(substituted C₁₋₆ alkyl)amino-C₂₋₆ alkyl, substituted C₁₋₆ alkylthio-C₂₋₆ alkyl, wherein the substituents are selected from the group consisting of pyrrolidino, piperidino, morpholino, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy-C₃₋₆ alkenyl)piperazino, 4-N—(C₁₋₆ alkylamino-C₁₋₆ alkyl)piperazino, 4-N—(C₁₋₆ alkylamino-C₃₋₆ alkenyl)piperazino, imidazolyl, oxazolyl, and thiazolyl;
(v) R₇—C(O) -C₁₋₆ alkyl or R₇—C(O) -C₂₋₆ alkenyl, in which R₇ is defined as above in [B(iv)];
(vi) HO—C₁₋₆ alkyl-C₂₋₆ alkenyl, R₇—O—C₁₋₆ alkyl-C₂₋₆ alkenyl, R₇NH—C₁₋₆ alkyl-C₂₋₆ alkenyl, R₆R₇N—C₁₋₆ alkyl-C₂₋₆ alkenyl, R₇NH—C(O)—O—C₁₋₆ alkyl-C₂₋₆ alkenyl, R₆R₇N—C(O)—O—C₁₋₆ alkyl-C₂₋₆ alkenyl, R₇O—C(O)—O—C₁₋₆ alkyl-C₂₋₆ alkenyl, or R₇—C(O)—O—C₁₋₆ alkyl-C₂₋₆ alkenyl, wherein R₆ and R₇ is defined as above in [B(iv)]; and
(vii) R₇—O—C₀₋₃ alkyl-C₃₋₆ cycloalk-1-yl, R₇NH— C₀₋₃ alkyl- C₃₋₆ cycloalk-1-yl, R₆R₇N— C₀₋₃ alkyl- C₃₋₆ cycloalk-1-yl, R₇NH—C(O)—O— C₀₋₃ C₃₋₆ cycloalk-1-yl, R₆R₇N—C(O)—O— C₀₋₃ alkyl- C₃₋₆ cycloalk-1-yl, R₇O— C(O)—O— C₀₋₃ alkyl- C₃₋₆ cycloalk-1-yl, R₇—C(O)—O—C₀₋₃ alkyl- C₃₋₆ cycloalk-1-yl, R₇O—C(O)—C₀₋₃ alkyl- C₃₋₆ cycloalk-1-yl, wherein R₇ and R₆ are defined as above in [B(iv)];

R₂ and R₃ are each independently selected from the group consisting of:
(viii) hydrogen, halo, trihalomethyl, C₁₋₆ alkyl, substituted C₁₋₆ alkyl, C₂₋₆ alkenyl, substituted C₂₋₆ alkenyl, C₁₋₆ alkyloxy, substituted C₁₋₆ alkyloxy, C₃₋₆ alkenyloxy, substituted C₃₋₆ alkenyloxy, C₁₋₆ alkylamino, substituted C₁₋₆ alkylamino, C₃₋₆ alkenylamino, or substituted C₃₋₆ alkenylamino; and
(ix) mono-, di-, or tri-substituted phenyl wherein the substituents are independently selected from the group consisting of:
(a) halo, trifluoromethyl, or substituted C₁₋₆ alkyl;
(b) C₁₋₆ alkyloxy, substituted C₁₋₆ alkyloxy, C₃₋₆ alkenyloxy, substituted C₃₋₆ alkenyloxy;
(c) C₁₋₆ alkyl-amino, di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, or di(substituted C₃₋₆ alkenyl)amino; and
(d) pyrrolidino, piperidino, morpholino, imidazolyl, substituted imidazolyl, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, or 4-N—(C₁₋₆ alkylamino C₃₋₆ alkenyl)piperazino;
   wherein the substituents for (a), (b), (c), and (d) are selected from the group consisting of:
(1) hydrogen, hydroxy, halo, or trifluoromethyl;
(2) C₁₋₆ alkylalkoxy, C₁₋₆ alkylamino, or C₁₋₆ alkylthio;
(3) C₃₋₆ alkenyloxy, C₃₋₆ alkenylamino, or C₃₋₆ alkenylthio; and
(4) pyrrolidino, piperidino, morpholino, imidazolyl, substituted imidazolyl, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, or 4-N—(C₁₋₆ alkylamino C₃₋₆ alkenyl)piperazino;

with the proviso that a) at least one of R₂ and R₃ is selected from [B (ix)] and wherein the substituents are selected from [B (ix) (b)-(d)] above; or b) R₂ and R₃ are taken together to form an optionally substituted aryl group, wherein the substituents are defined as above in [B (ix) (a)-(d)];

and R₄ is selected from the group consisting of:
(i) hydrogen;
(ii) substituted C₁₋₁₁ alkyl or C₂₋₁₁ alkenyl wherein the substituents are independently selected from the group consisting of:
   (a) hydrogen, hydroxy, C₁₋₆ alkyloxy, C₁₋₆alkylthio, C₁₋₆ alkylamino, phenyl-C₁₋₆ alkylamino, or C₁₋₆ alkoxycarbonyl;
   (b) substituted C₁₋₆ alkyloxy, C₃₋₆ alkenyloxy, or substituted C₃₋₆ alkenyloxy;
   (c) di(C₁₋₆alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, or di(substituted C₃₋₆ alkenyl)amino; and (d) pyrrolidino, piperidino, morpholino, imidazolyl, substituted imidazolyl, piperazino, 4-N— C₁₋₆ alkylpiperazino, 4-N—C₃₋₆alkenylpiperazino, 4-N—(C₁₋₆alkoxy C₁₋₆ alkyl)piperazino, 4-N—(C₁₋₆ alkoxy C₃₋₆ alkenyl)piperazino, 4-N—(C₁₋₆alkylamino C₁₋₆ alkyl)piperazino, or 4-N—(C₁₋₆alkylamino C₃₋₆ alkenyl)piperazino; and
(iii) aryl C₀₋₁₁ alkyl wherein the aryl group is selected from phenyl, imidazolyl, furyl, or thienyl.

In some embodiments of the invention, the compound of Formula 1a is a compound wherein R₁ is selected from the group consisting of mono-, di-, and tri-substituted aryl-Cₘ alkyl wherein aryl is selected from the group consisting of phenyl and thienyl, and the substituents are selected from the group consisting of:
(a) C₁₋₆ CO₂R₅, trans-CH═CHCO₂R₅, C₁₋₆CONHR₅, or trans-CH═CHCONHR₅;
(b) C₁₋₆CONR₆R₇, or trans-CH═CHCONR₆R₇;
(c) R₇ C(O) C₁₋₆ alkyl or R₇ C(O) C₂₋₆ alkenyl; and
(d) HO—C₁₋₆ alkyl-C₂₋₆ alkenyl, R₇—O—C₁₋₆ alkyl-C₂₋₆ alkenyl, R₇NH—C₁₋₆alkyl-C₂₋₆ alkenyl, R₆R₇N—C₁₋₆alkyl-C₂₋₆alkenyl, R₇NH—C(O)—O—C₁₋₆alkyl-C₂₋₆alkenyl,

R₆R₇N—C(O)—O—C₁₋₆alkyl-C₂₋₆alkenyl, R₇O—C(O)—O—C₁₋₆alkyl-C₂₋₆alkenyl, or R₇—C(O)—O—C₁₋₆ alkyl-C₂₋₆ alkenyl.

In other embodiments, the compound of Formula 1a is a compound wherein R₁ is selected from the group consisting of mono-, di-, and tri-substituted aryl-Cₘ alkyl wherein aryl is selected from the group consisting of phenyl and thienyl, and the substituents are selected from the group consisting of:
(a) C₁₋₆ CO₂R₅, trans-CH═CHCO₂R₅, C₁₋₆CONHR₅, or trans-CH═CHCONHR₅;
(b) C₁₋₆CONR₆R₇, or trans-CH═CHCONR₆R₇;
(c) R₇ C(O) C₁₋₆ alkyl or R₇ C(O) C₂₋₆ alkenyl; and
(d) HO—C₁₋₆ alkyl-C₂₋₆ alkenyl, R₇—O—C₁₋₆ alkyl-C₂₋₆ alkenyl, R₇NH—C₁₋₆alkyl-C₂₋₆ alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆alkyl-C₂₋₆alkenyl, R₇O—C(O)—O—C₁₋₆alkyl-C₂₋₆alkenyl, or R₇—C(O)—O—C₁₋₆ alkyl-C₂₋₆ alkenyl.

In various embodiments of the invention, the compound of Formula 1a is a compound wherein R₁ is selected from the group consisting of mono-, di-, and tri-substituted aryl-C₀₋₆ alkyl wherein aryl is selected from the group consisting of phenyl and thienyl, and the substituents are HO—C₁₋₆ alkyl-C₂₋₆ alkenyl, R₇—O—C₁₋₆ alkyl-C₂₋₆ alkenyl, R₇NH—C₁₋₆ alkyl-C₂₋₆ alkenyl, R₆R₇N—C₁₋₆ alkyl-C₂₋₆ alkenyl, R₇NH—C(O)—O—C₁₋₆ alkyl-C₂₋₆ alkenyl, R₆R₇N—C(O)—O—C₁₋₆ alkyl-C₂₋₆ alkenyl, R₇O—C(O)—O—C₁₋₆ alkyl-C₂₋₆ alkenyl, or R₇—C(O)—O—C₁₋₆ alkyl-C₂₋₆ alkenyl.

In other embodiments, R₁ is selected from the group consisting of mono-, di-, and tri-substituted aryl-C₀₋₆ alkyl wherein the aryl-C₀₋₆ alkyl is phenyl-C₀₋₆ alkyl. In some embodiments, R₁ is selected from the group consisting of mono-, di-, and tri-substituted aryl-C₀₋₆ alkyl wherein the aryl-C₀₋₆ alkyl is aryl-C₀alkyl, which is aryl with no alkyl group attached directly to aryl.

In various embodiments, R₂ and R₃ are each independently selected from the group consisting of: mono-, di-, and tri-substituted phenyl wherein the substituents are independently selected from the group consisting of:
(i) C₁₋₆ alkyloxy, substituted C₁₋₆ alkyloxy, C₃₋₆ alkenyloxy, or substituted C₃₋₆ alkenyloxy;
(ii) C₁₋₆ alkyl-amino, di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, or di(substituted C₃₋₆ alkenyl)amino, and
(iii) pyrrolidino, piperidino, morpholino, imidazolyl, substituted imidazolyl, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, or 4-N—(C₁₋₆ alkylamino C₃₋₆ alkenyl)piperazino.

In some embodiments, R₂ and R₃ are each independently selected from the group consisting of: mono-, di-, and tri-substituted phenyl wherein the substituents are independently selected from the group consisting of C₁₋₆ alkyl-amino, di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, and di(substituted C₃₋₆ alkenyl)amino. In some embodiments, R₄ is hydrogen.

In some embodiments, the compound of Formula 1a is a compound of Formula 1b:

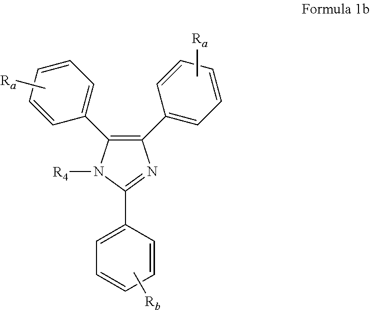

wherein each instance of Rₐ is independently C₁₋₆ alkyl-amino, di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, or di(substituted C₃₋₆ alkenyl)amino; and

R_{b} is HO—C₁₋₆ alkyl-C₂₋₆ alkenyl, R₇—O—C₁₋₆ alkyl-C₂₋₆ alkenyl, R₇NH—C₁₋₆ alkyl-C₂₋₆ alkenyl, R₆R₇N—C₁₋₆ alkyl-C₂₋₆ alkenyl, R₇NH—C(O)—O—C₁₋₆ alkyl-C₂₋₆ alkenyl, R₆R₇N—C(O)—O—C₁₋₆ alkyl-C₂₋₆ alkenyl, R₇O—C(O)—O—C₁₋₆ alkyl-C₂₋₆ alkenyl, or R₇—C(O)—O—C₁₋₆ alkyl-C₂₋₆ alkenyl.

In some embodiments, the compound of Formula 1 or 1a (such as a compound of Formula 1b or 2), is in the form of a free compound or as its pharmaceutically-acceptable pro-drug, metabolite, analogue, derivative, solvate or salt, and is selected from the group consisting of: (2[4-(3-ethoxy-1-propenyl)phenyl]-4,5-bis(4-(2-propylamino)phenyl)-1H-imidazole; 2-[4-(3-ethoxy-trans-1-pro-pen-1-yl)phenyl]-4,5-bis(4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2[4-(3-methoxy-trans-1-propen-1-yl)ph-enyl]-4,5-bis(4-pyrrolidinophenyl) imidazole; 2-[4-(3-ethoxy-trans-1-prop-en-1-yl)phenyl]-4,5-bis(4-pyrrolidinophenyl) imidazole; 2-[4-(3-ethoxy-trans-1-propen-1-yl)phenyl]-4-(4-N-dimethylaminophenyl)-5 (4 pyrrolidinophenyl) imidazole; 2[4-(3-ethoxy-trans-1-propen-1-yl)phenyl-]-4-(4-N methylaminophenyl)-5-(4-pyrrolidino-phenyl) imidazole; 2-[4-(3-ethoxy-trans-1-propen-1-yl)phenyl]-4,5-bis(4-N-morpholinophenyl) imidazole; 2-[4-(3-ethoxy-trans-1-propen-1-yl)phenyl]-4-(4-N-dimethylamin-ophenyl)-5-(4-N-morpholinophenyl) imidazole; 2-[4-(3-ethoxy-trans-1-propen 1 yl)phenyl]-4-(4-N-methylaminophenyl)-5-(4-N-morpholinophenyl) imidazole; and 2[4-(3-ethoxy-trans-1-propen-1-yl)phenyl]-4-4-N methylami-nophenyl)-5-(4-N-isopropylaminophenyl) imidazole.

The compound of Formula 1 or 1a can be the specific formulas as described in U.S. Pat. Nos. 5,700,826 and 5,840,721, herein incorporated by reference. Preferred compositions and methods comprise the compound of the following formula (Formula 2):

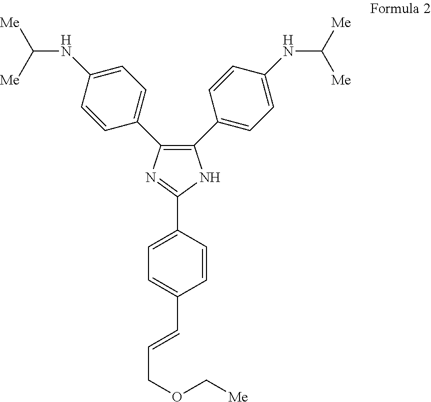 [SMILES: CCOC/C=C/c1ccc(-c2nc(-c3ccc(NC(C)C)cc3)c(-c3ccc(NC(C)C)cc3)[nH]2)cc1]

in the form of a free compound or as its pharmaceutically-acceptable pro-drug, metabolite, analogue, derivative, solvate or salt.

In another related embodiment the agent is an MDR inhibitor and the MDR inhibitor is chosen from the group consisting of: calcium channel blockers, calmodulin inhibitors, antibiotica, cardiovascular agents, noncytotoxic analogs of anthracyclines and vinca alkaloids, cyclosporine A, FK-506, and derivatives of cyclopeptides.

In a second aspect the invention provides a method of treating a condition characterized by altered lipid metabolism by administering an agent that modulates α-synuclein. Agents that can modulate α-synuclein can be selected from but not limited to those presented in Table 1. In certain embodiments modulation can include but not be limited to altered fibrillation, folding, ubiquitination, trafficking, synaptic targeting, lysosomal storage, expression, subcellular compartmentalization, and lipid-interactions. In one embodiment the altered lipid metabolism is in astrocytes. In one embodiment the altered lipid metabolism is an accumulation of glucocerebroside. In another embodiment the condition is selected from the group consisting of: Gaucher disease, Fabry disease, lysosomal storage diseases, lipid storage diseases, glycoprotein storage diseases, mucolipidoses, gangliosidoses, leukodystrophies, mucopolysaccharidoses, Niemann-Pick disease, Tay Sachs diseases, Hunter syndrome, Hurler disease, Sandhoff's disease and cystic fibrosis. In specific embodiments the agent that corrects α-synuclein dysfunction is selected from apomorphine, pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, norepinephrine, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, benserazide hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′,4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3′-dihydroxyflavone, 3′,4′-dihydroxyflavone, 5,6-dihydroxy-7-methoxyflavone, baicalein-7-methyl ether, 1-dopa, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin, derivatives and analogs thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. Depicts the in vitro Conduritol B epoxide treatment paradigm in SH-SY5Y cells.

FIG. 2. Depicts the in vivo Conduritol B epoxide administration paradigm in C57B1/6 mice.

FIG. 3. α-synuclein in neuroblastoma cells. (A) Differentiated SH-SY5Y cell were exposed to increasing concentrations of the GCase inhibitor CBE for 48 h. Western blot analysis showed increased levels of α-synuclein in cell treated with 50-200 μM CBE-vs. vehicle-treated cells. (B) Expression of α-synuclein mRNA was measured using RT-PCR in differentiated SH-SY5Y cells exposed to increasing concentrations of CBE for 48 h. No change in α-synuclein transcription was detected.

FIG. 4. α-synuclein in ventral mesencephalon. C57BL/6 mice were administered a single dose of 200 mg/kg CBE or DMSO and sacrificed at 48 h. Western blot analysis of ventral mesencephalon samples showed an increase in α-synuclein levels in the P1 fraction of CBE-vs. DMSO-treated mice, with no change in the S1 fraction.

FIG. 5. α-synuclein in mouse brain. C57BL/6 mice were administered a single dose of 200 mg/kg CBE or DMSO and sacrificed at 48 h. (A) Immunohistochemical experiments revealed an apparent increase in the levels of GFAP (Cy3) and α-synuclein (FITC) in the substantia nigra of CBE-vs. vehicle-treated mice. Bar=50 μm. (B) No alteration in protein levels were detected in cortex. Bar=20 μm.

FIG. 6. Neuronal and glia α-synuclein the substantia nigra. Analyses at higher magnification clearly demonstrate accumulation of α-synuclein (FITC) within cell bodies and processes in the substantia nigra of CBE-vs. vehicle-treated mice. Glial activation was robust within neuropil, as shown by GFAP immunohistochemistry (Cy3), and colocalization of α-synuclein immunoreactivity was observed within astrocytes of CBE-treated mice (arrows). Hoescht staining (DAPI) was used to identify nuclei. Bar=20 μm.

FIG. 7. Increased α-synuclein expression within the substantia nigra in aged mice treated subchronically with CBE vs. DMSO.

FIG. 8. Accumulation of silver grains in nigral neurons from CBE- but not DMSO-treated mice.

FIG. 9. α-synuclein alterations exist in brain from Parkinson's Disease patients who carry a Gaucher mutation. Pictured is a Western blot analysis of α-synuclein of samples from a Gau+/− brain.

### DETAILED DESCRIPTION OF THE INVENTION

The present invention describes methods of modulating α-synuclein function, lipid metabolism and lysosomal storage by using agents that modulate α-synuclein function, lysosomal storage and lipid metabolism, in particular glycosphingolipid metabolism.

I. α-Synuclein-Related Disorders

Synucleins are a family of small, presynaptic neuronal proteins composed of α-, β-, and γ-synucleins, of which only α-synuclein aggregates have been associated with several neurological diseases (Ian et al., Clinical Neurosc. Res. 1:445-455, 2001; Trojanowski and Lee, Neurotoxicology 23:457-460, 2002). The role of synucleins (and in particular, α-synuclein) in the etiology of a number of neurodegenerative and/or amyloid diseases has developed from several observations. Pathologically, α-synuclein was identified as a major component of Lewy bodies, the hallmark inclusions of Parkinson's disease, and a fragment thereof was isolated from amyloid plaques of a different neurological disease, Alzheimer's disease. Biochemically, recombinant α-synuclein was shown to form amyloid-like fibrils that recapitulated the ultrastructural features of α-synuclein isolated from patients. α-synuclein-related pathology is involved in the etiology of a variety of neurological disorders, including Parkinson's Disease, Parkinson's Disease with accompanying dementia, Lewy body dementia, Lewy body variant of Alzheimer's disease, Huntington's disease, Alzheimer's disease with Parkinsonism, and multiple system atrophy. Abnormal protein aggregates are a common pathological feature of many neurodegenerative diseases.

α-synuclein may be the biological link between diseases such as Gaucher and Parkinson's diseases and is the basis of the invention described herein. α-synuclein pathology is common to several neurodegenerative diseases. Gene multiplications cause a severe and rapidly progressive parkinsonism (Singleton et al. 2003). Changes in α-synuclein levels are associated with increased neuronal vulnerability (Vila et al. 2000; Manning-Bog et al. 2002; Sherer et al. 2003). Lysosomal degradation is a major clearance mechanism for α-synuclein from cells (Lee et al. 2004); this and other pathways may be affected by abnormal glucocerebrosidase (Hruska et al. 2006; Goker-Alpan et al. 2006). α-synuclein directly interacts with glucocerebroside-containing lipids: the protein strongly binds human-derived glucosylceramide (Schlossmacher et al. 2005).

II. Agents Useful for Modulating α-Synuclein

Agents that can modulate α-synuclein can be selected from but not limited to those presented in Table 1. In certain embodiments modulation can include but not be limited to altered fibrillation, folding, ubiquitination, trafficking, synaptic targeting, lysosomal storage, expression, subcellular compartmentalization, and lipid-interactions.

TABLE 1
Compounds that modulate α-synuclein function

| | | |
|---|---|---|
| apomorphine | cefamandole sodium | fisetin |
| pyrogallol | cephaloridine | luteolin |
| 1,4-naphthoquinone | myricetin | fustin |
| cisplatin | 6,2′,3′-trihydroxyflavone | epicatechin gallate |
| isoproterenol | 5,7,3′,4′,5′-pentahydroxyflavone | catechin |
| pyrogallin | 7,3′,4′,5′-tetrahydroxyflavone | alizarin |
| cianidanol | (5,6,7,4′-tetrahydroxyflavone) | tannic acid |
| sulfasalazine | baicalein | eriodyctol |
| quinalizarin | eriodictyol | carboplatin |
| benserazide | 7,3′,4′-trihydroxyisoflavone | purpurogallin-4-carboxylic acid |
| hexachlorophene | epigallocatechin gallate | koparin |
| pyrvinium pamoate | quercetin | 2,3,4-trihydroxy-4′- |
| | | ethexybenzophenone |
| dobutamine | gossypetin (3,5,7,8,3′,4′- | baeomycesic acid |
| | hexahydroxyflavone) | |
| methyl-dopa | 2′,3′-dihydroxyflavone | hamtoxylin |
| curcumin | 3′,4′-dihydroxyflavone | iriginol hexaaceatate |
| berberine chloride | 5,6-dihydroxy-7- | 4-acetoxyphenol |
| | methoxyflavone | |
| daidzein | baicalein-7-methyl ether | theaflavin monogallate |
| merbromin | Levodopa (L-Dopa) | theaflavin digallate |
| norepinephrine | DOPAC | stictic acid |
| dopamine hydrochloride | homogentisic acid | purpurogallin |
| carbidopa | 6-hydroxydopamine | 2,5-dihydroxy-3,4-dimethoxy-4′- |
| | | ethoxybenzophenone |
| ethylnorepinephrine hydrochloride | epinephrine | promethazine hydrochloride |
| tannic acid | 3,4-dihydroxycinnamic acid | oxidopamine hydrochloride |
| elaidyphosphocholine | 2,3-dihydroxynaphthalene | pyrantel pamoate |
| hydroquinone | 3,4-dihydroxybenzoic acid | elaidylphosphocholine |
| chlorophyllide Cu complex Na salt | 3,4,5-trihydroxybenzoic acid | amphotericin B |
| methyldopa | 1,2,3-trihydroxybenzoic acid | gallic acid |
| isoproterenol hydrochloride | gallate (gallic acid) | fumarprotocetraric acid |
| benserazide hydrochloride | benzoquinone | theaflavin |
| dopamine | catechol | haematoxylin pentaacetate |
| dobutamine hydrochloride | rifampicin | 4-methoxydalbergione |
| thyroid hormone | rosmarinic acid | epigallocatechin-3-monogallate |
| purpurin | baicalin | rolitetracycline |
| sodium beta-nicotinamide adenine | tanshinones I and II | 7,3′-dimethoxyflavone |
| dinucleotide phosphate | | |
| lansoprazole | emodin | liquiritigenin dimethyl ether |
| dyclonine hydrochloride | procyanidin B4 | catechin pentaacetate |
| pramoxine hydrochloride | resveratrol | apigenin |
| azobenzene | rutin | 3,4-dedesmethyl-5-deshydroxy-3′- |
| | | ethoxyscleroin |

III. Lipid Metabolism and Lipid Storage Disorders

a. Glycosphingolipid Metabolism (GSL) and Lysosomal Storage Disorders

The importance of treating GSL metabolism disorders is underscored by various important roles sphingolipids have. Sphingolipids are ubiquitous constituents of membrane lipids in mammalian cells. Sphingolipids are involved in membrane trafficking and intracellular signaling as a factor requiring for the formation of membrane micro domains so called lipid rafts. In addition to being the building blocks of biological membranes, glycosphingolipids appear to be involved in cell proliferation (Hannun and Bell, Science, 243:500-507 (1989)) differentiation (Schwarz et al., J. Biol. Chem. 270:10990-10998 (1995); Harel and Futerman, J. Biol. Chem. 268:14476-14481 (1993)), oncogenic transformation (Hakomori, Annu. Rev. Biochem. 50:733-764 (1981); Morton et al., Prog. Brain Res. 101:251-275 (1994)) and the prevention of the onset of apoptosis (Nakamura et al., J. Biol. Chem. 271:1255-1257 (1996)).

The biosynthesis process of sphingolipids is as follows: the first step is the condensation reaction of L-serine with palmitoyl CoA. The reaction is catalyzed by serine palmitoyl transferase to generate 3-ketodihydrosphingosine. The resulting 3-ketodihydrosphingosine is then reduced to dihydrosphingosine. The obtained dihydrosphingosine can then undergo N-acylation followed by desaturation to generate ceramide (Cer). These reactions to produce Cer typically occur on the cytosolic surface of the endoplasmic reticulum (ER). Cer is then thought to be delivered to the lumenal side of the Golgi apparatus and converted to sphingomyelin (SM) by SM synthase catalyzing transfer of phosphocholine from phosphatidylcholine (PC) to Cer. Cer is also converted to glucosylceramide (GlcCer). Glucosylceramides are produced by glucosylceramide synthase (GCS) transferring glucose from UDP-glucose to ceramide (Basu, et al., (1968) J. Biol. Chem 243:5802-5804). The rate of GlcCer formation under physiological conditions usually depends on the tissue level of UDP-glucose, which in turn depends on the level of glucose in a particular tissue (Zador et al., J. Clin. Invest. 91:797-803 (1993)). In vitro assays based on endogenous ceramide typically yield lower synthetic rates than mixtures containing added ceramide, suggesting that tissue levels of ceramide are also normally rate-limiting (Brenkert et al., Brain Res. 36:183-193 (1972)).

However, unlike many other GSLs, GlcCer is typically made on the outer leaflet of the Golgi bilayer (Lannert et al., J. Biol. Chem 273:2939-2946 (1998)). As a result, for GlcCer to be accessed by glycosyltransferases for further carbohydrate elongations, GlcCer typically needs to be translocated, or “flipped”, into the lumen of the Golgi. MDR1 can function as a glycolipid flippase and appears to be responsible for the translocation of GlcCer into the lumen for further carbohydrate elongation. MDR1 translocation appears to be specific for natural GSL synthesis (DeRosa et al., J. Biol. Chem. 279:7867-7876 (2004)). Compounds of the present invention can specifically inhibit the translocation or flippase function of MDR1, or may be specific for modulating neutral GSL synthesis, acidic GSL synthesis, or both. For example, the compound can inhibit Gb3 accumulation but not gangliosides, whereas other compounds inhibit accumulation of both Gb3 and gangliosides.

Most glycosphingolipids (GSLs) are derived from glucosylceramide (GlcCer). GSLs are a subtype of glycolipids containing the amino alcohol sphingosine, and include cerebrosides, gangliosides, and globosides. Cerebrosides are important components of animal and muscle nerve cells, and include myelin. Gangliosides are GSLs with one or more sialic acids, common gangliosides being GD1a, GD1b, GD2, GD3, GM1, GM2, GM3, and GT1b. Gangliosides are a component of the plasma membrane and modulate cell signal transduction events. They are also present in lipid rafts. Globosides are GSLs with N-acetylgalactosamine as the side chain. Sphingomyelin is present in animal cell membranes and may have a role in signal transduction. Defects in the metabolism of GSLs can lead to different diseases, for example, a defect in the degradation of glucocerebrosides can cause Gaucher's, defect in galactocerebrosides can cause Karbbe disease. Gangliosides are important in immunology and may be involved in neurodegenerative diseases. Defects in β-hexosadminidase, which cleaves the side chain of globosides and gangliosides, can lead to Sandhoff disease, and sphingomyelin accumulation can lead to Niemann-Pick disease. In other embodiments diseases can include Parkinson's disease, Parkinson's disease with accompanying dementia, Lewy body dementia, Lewy body variant of Alzheimer's disease, Huntington's disease, Alzheimer's disease with Parkinsonism, and multiple system atrophy.

The compositions and methods described herein are effective in treating GSL metabolic conditions or α-synuclein-mediated conditions in which GSL metabolism is altered. In some aspects, conditions due to any defective enzyme, or abnormal levels of substrates/products of the GSL biosynthesis pathways, may be treated. Conditions include Gaucher (GlcCer accumulation) and Fabry (globotraiosyl, or Gb3, accumulation), as well as other lysosomal storage diseases including, but not limited to, Niemann-Pick, Tay Sachs, and Sandhoff's disease. Other diseases with impaired glycosylated proteins, such as cystic fibrosis can also be treated by compositions and methods of the present invention.

Many known lysosomal storage diseases (LSDs) involve a similar pathogenesis, namely, a compromised lysosomal hydrolase. Generally, LSDs result from genetic deficiencies in glycoconjugate catabolism, which may be due to the activity of a single lysosomal hydrolytic enzyme, such as a specific lysosomal sugar hydrolase or its activator protein, being reduced or lacking altogether. The substrate of the compromised enzyme accumulates undigested in lysosomes, producing severe disruption of cellular architecture and various disease manifestations. A number of sphingolipidoses, or types of LSDs, caused by deficient activity of lysosomal enzymes crucial for the degradation of sphingolipids, is shown in Table 2, and may be treated by the compositions and methods of the present invention. For example, in “glycosphingolipidoses,” ccumulation typically results in the formation of lipid inclusions and multilamellar structures that prevent normal cell functions. LSDs can be classified by the nature of their storage material, such as lipid storage disorders (including Gaucher's and Nieman-Pick), gangliosidoses (such as Tay-Sachs disease), leukodystrophies, mucopolysaccharidoses (including Hunter syndrome and Hurler disease), glycoprotein storage disorders, and mucolipidoses.

Gaucher's disease is one of the most common lysosomal storage diseases known. Type 1 is usually the most common among three recognized clinical types and typically follows a chronic course which does not involve the nervous system. Types 2 and 3 both have a CNS component, the former typically being an acute infantile form with death by age two and the latter a subacute juvenile form. The incidence of Type 1 Gaucher's disease is about one in 50,000 live births and about one in 400 live births among Ashkenazis (Kolodny et al., 1998, “Storage Diseases of the Reticuloendothelial System”, In: Nathan and Oski's Hematology of Infancy and Childhood, 5th ed., vol. 2, David G. Nathan and Stuart H. Orkin, Eds., W. B. Saunders Co., pages 1461-1507). Also known as glucosylceramide lipidosis, Gaucher's disease is typically caused by inactivation of the enzyme glucocerebrosidase and accumulation of glucocerebroside (also known as GlcCer). Glucocerebrosidase normally catalyzes the hydrolysis of glucocerebroside to glucose and ceramide. In Gaucher's disease, glucocerebroside accumulates in tissue macrophages which become engorged. These cells are typically found in liver, spleen and bone marrow and occasionally in lung, kidney and intestine. Secondary hematologic sequelae include severe anemia and thrombocytopenia in addition to the characteristic progressive hepatosplenomegaly and skeletal complications, including osteonecrosis and osteopenia with secondary pathological fractures.

Niemann-Pick disease, also known as sphingomyelin lipidosis, comprises a group of disorders characterized by foam cell infiltration of the reticuloendothelial system. Foam cells in Niemann-Pick become engorged with sphingomyelin and, to a lesser extent, other membrane lipids including cholesterol. Niemann-Pick is typically caused by inactivation of the enzyme sphingomyelinase in Types A and B disease, with 27-fold more residual enzyme activity in Type B. The pathophysiology of major organ systems in Niemann-Pick can be briefly summarized as follows. The spleen is the most extensively involved organ of Type A and B patients. The lungs are involved to a variable extent, and lung pathology in Type B patients is the major cause of mortality due to chronic bronchopneumonia. Liver involvement is variable, but severely affected patients may have life-threatening cirrhosis, portal hypertension, and ascites. The involvement of the lymph nodes is variable depending on the severity of disease. Central nervous system (CNS) involvement differentiates the major types of Niemann-Pick. While most Type B patients do not experience CNS involvement, it is characteristic in Type A patients. The kidneys are only moderately involved in Niemann Pick disease.

Fabry disease is an X-linked recessive LSD characterized by a deficiency of α-galactosidase A (α-Gal A), also known as ceramide trihexosidase, which leads to vascular and other disease manifestations via accumulation of glycosphingolipids with terminal α-galactosyl residues, such as globotriaosyl ceramide (GL-3, or Gb3) (see generally Desnick R J et al., 1995, α-galactosidase A Deficiency: Fabry Disease, In: The Metabolic and Molecular Bases of Inherited Disease, Scriver et al., eds., McGraw-Hill, New York, 7.sup.th ed., pages 2741-2784). Symptoms may include anhidrosis (absence of sweating), painful fingers, left ventricular hypertrophy, renal manifestations, and ischemic strokes. The severity of symptoms varies dramatically (Grewal, J. Neurol. 241:153-15 (1994)). A variant with manifestations limited to the heart is recognized, and its incidence may be more prevalent than once believed (Nakao. N. Engl. J. Med. 333:288-293 (1995)).

Tay-Sachs disease, also known as GM2 gangliosidosis or hexosaminidase A deficiency, is a genetic disorder wherein the most common variant, infantile Tay-Sachs disease, is fatal. The disease is typically caused by mutations on the HEXA gene. The HEXA gene encodes the α-subunit of the lysosomal enzyme β-hexosaminidase A. Hydrolysis of GM2-ganglioside typically requires three proteins. Two subunits of hexosaminidase A, and a small glycolipid transport protein, the GM2 activator protein (GM2A), which acts as a substrate specific cofactor for the enzyme. Deficiency in any one of these proteins leads to storage of the ganglioside, primarily in the lysosomes of neuronal cells lysosomes of neuronal cells. Deficiencies in hexosaminidase A caused by HEXA mutations can lead to Tay-Sachs disease.

Patients with Sandhoff's disease have similar symptoms to Tay-Sachs. Sandhoff's is a lipid storage disorder that causes progressive destruction of nerve cells. The disease is typically inherited and involves the CNS and mutations in the HEXB gene which encodes the β-subunit of the lysosomal enzymes β-hexosaminidase A and B. Thus, HEXB mutations can affect both β-hexosaminidase A and B and prevent breakdown of GM2 gangliosides and other molecules leading to accumulation of these molecules, causing nerve cell destruction and disease.

Diseases and conditions other than LSDs are also treated by the compositions and methods of the present invention. For example, other diseases resulting from, or which result in, increased glycosphingolipid synthesis can be treated, such as cystic fibrosis. Cystic fibrosis (CF) epithelial cells express a greater density of an asialylated ganglioside (gangliotetraosyl ceramide, Gg4), on their apical surface, which manifest as a higher susceptibility of CF individuals of acquiring bacterial infections. (Hart and Winstanley, British Medical Bulletin 61:81-96 (2002)).

TABLE 2
Major Sphingolipidoses

| Clinical diagnosis | Affected lipids | Enzyme defect |
|---|---|---|
| GM1 gangliosidosis | GM1 ganglioside | β-Galactosidase |
| | Galactose-rich fragments of | |
| | glycoproteins | |
| GM2 gangliosidosis | GM2 ganglioside | β-Hexosaminidase A |
| Tay-Sachs disease, B variant | | |
| B1 variant | GM2 ganglioside | β-Hexosaminidase |
| AB variant | GM2 ganglioside | GM2 activator protein |
| Sandhoff's disease, O variant | GM2 ganglioside | β-Hexosaminidase A, B |
| | Asialo GM2 ganglioside, Globoside | |
| Niemann-Pick disease (A and B) | Sphingomyelin | Sphingomyelinase |
| Gaucher's disease | Glucosylceramide | Glucosylceramidase |
| | Glucosylsphingosine | |
| Farber's disease | Ceramide | Acid ceramidase |
| Fabry's disease | Trihexosylceramide | a-Galactosidase A |
| Metachromatic leukodystrophy | Sulfatide | Arylsulfatase A |
| Multiple sulfatase deficiency | Sulfatide and other compounds | Arylsulfatase A, B, C and others |
| Globoid cell leukodystrophy | Galactosylceramide | Galactosylceramidase |
| (Krabbe's disease) | Galactosylsphingosine | |
| Total SAP deficiency | Multiple sphingolipids | Sphingolipid activator protein |
| SAP-B deficiency | Sulfatide and others | Sulfatidase activator (SAP-B) |
| SAP-C deficiency | Glucosylceramide | SAP-C |

IV. Agents Useful for Altering Lipid Metabolism

In certain embodiments this disclosure provides compounds for altering lipid metabolism such as agents that modulate glycosphingolipid levels such as MDR inhibitors, compounds that increase glucocerebrosidase levels, and cholesterol lowering drugs such as statins.

a. Agents Useful for Modulating Glycosphingolipid (GSL) Levels

i. Imidazole Derivative or Compounds

The class of imidazole derivatives or compounds is as depicted in Formula 1:

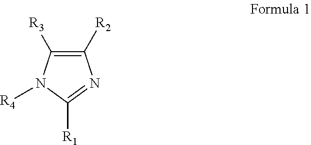

in the form of a free compound or its pharmaceutically acceptable pro-drug, metabolite, analogue, derivative, solvate or salt wherein the substituents R₁, R₂, R₃, and R₄ are defined as described in (a) and (b) below:

(a) when R₁ is selected from the group consisting of:

(i) substituted C₁₋₁₁ alkyl or substituted C₂₋₁₁ alkenyl, wherein the substituents are selected from the group consisting of hydroxy, C₁₋₆ alkyloxy; or

(ii) mono-, di-, and tri-substituted aryl-C₀₋₁₁ alkyl wherein aryl is selected from the group consisting of phenyl, furyl, thienyl wherein the substituents are selected from the group consisting of:
(a) phenyl, trans-2-phenylethenyl, 2-phenylethynyl, 2-phenylethyl, wherein the said phenyl group is mono- or disubstituted with a member selected from the group consisting of hydroxy, halo, C₁₋₄ alkyl and C₁₋₄ alkyloxy,
(b) substituted C₁₋₆ alkyl, substituted C₂₋₆ alkyloxy, substituted C₂₋₆ alkylthio, substituted C₂₋₆ alkoxycarbonyl, wherein the substituents are selected from the group consisting of C₁₋₆ alkoxy, and C₁₋₆ alkylthio; and
(c) C₁₋₁₁ CO₂R₅, C₁₋₁₁CONHR₅, trans-CH═CHCO₂R₅, or trans-CH═CHCONHR₅ wherein R₅ is C₁₋₁₁ alkyl, or phenyl C₁₋₁₁ alkyl, C₁₋₆ alkoxycarbonylmethyleneoxy;

then R₂ and R₃ are each independently selected from the group consisting of mono-, di, and tri-substituted phenyl wherein the substituents are independently selected from:
(i) substituted C₁₋₆ alkyl,
(ii) substituted C₁₋₆ alkyloxy, C₃₋₆ alkenyloxy, substituted C₃₋₆ alkenyloxy,
(iii) substituted C₁₋₆ alkyl-amino, di(substituted C₁₋₆ alkyl)amino,
(iv) C₃₋₆ alkenyl-amino, di(C₃₋₆ alkenyl)amino, substituted C₃₋₆ alkenyl-amino, di(substituted C₃₋₆ alkenyl)amino,
(vi) pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, 4-N—(C₁₋₆ alkylamino C₃₋₆ alkenyl)piperazino,
wherein the substituents are selected from the group consisting of:
   (a) hydroxy, C₁₋₆ alkylalkoxy, C₁₋₆ alkylamino
   (b) C₃₋₆ alkenyloxy, C3-6 alkenylamino, or
   (c) pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, 4-N—(C₁₋₆ alkylamino C₃₋₆ alkenyl)piperazino,

or R₂ and R₃ taken together forming an aryl group or substituted aryl, wherein the substituents are defined as above in (i)-(v);

and R₄ is selected from the group consisting of:
(i) hydrogen;
(ii) substituted C₁₋₁₁ alkyl or C₂₋₁₁ alkenyl wherein the substituents are independently selected from the group consisting of hydrogen, hydroxy, C₁₋₆ alkyloxy, C₁₋₆alkylthio, C₁₋₆ alkylamino, phenyl-C₁₋₆ alkylamino, C₁₋₆ alkoxycarbonyl; or
(iii) substituted aryl C₀₋₁₁ alkyl wherein the aryl group is selected from phenyl, imidazolyl, furyl, thienyl in which the substituents are selected from A(a-c); or

(b) when R₁ is selected from the group consisting of:

Mono-, di-, and tri-substituted aryl-C₀₋₆ alkyl wherein aryl is selected from the group consisting of phenyl, thienyl, and the substituents are selected from the group consisting of:
(a) trans-2-substituted benzimidazolylethenyl, trans-2-substituted benzoxazolylethenyl, trans-2-substituted benzthiazolylethenyl, in which the substituents are selected from the group consisting of hydrogen, hydroxy, halo, trihalomethyl, C₁₋₄ alkyl and C₁₋₄ alkyloxy, C₁₋₄ alkyloxycarbonyl, C₁₋₄ alkylamino, di(C₁₋₄ alkyl)amino, C₃₋₆ alkenylamino, di(C₃₋₆alkenyl)amino, C₁₋₄ alkyloxy-C₁₋₄ alkylamino, substituted C₁₋₄ alkyl and C₁₋₄ alkyloxy, substituted C₁₋₄ alkyloxycarbonyl, substituted C₁₋₄ alkylamino, di(substituted C₁₋₄ alkyl)amino, substituted C₃₋₆ alkenylamino, di(substituted C₃₋₆ alkenyl)amino, wherein the substituents are as defined above,
(b) trans-2-cyano ethenyl, trans-2-alkylsulfonyl ethenyl, trans-2-alkenylsulfonyl ethenyl, trans-2-substituted alkylsulfonyl ethenyl, trans-2-substituted alkenylsulfonyl ethenyl, in which the substituents are defined above,
(c) C₁₋₆ CO₂R₅, trans-CH═CHCO₂R₅, C₁₋₆CONHR₅, or trans-CH═CHCONHR₅, wherein R₅ is C₁₋₆ alkoxy C₂₋₆ alkyl, amino C₂₋₆ alkyl, C₁₋₆ alkylamino C₂₋₆ alkyl, di(C₁₋₆alkyl)amino C₂₋₆ alkyl, C₁₋₆ alkylthio C₂₋₆ alkyl, substituted C₁₋₆ alkoxy C₂₋₆ alkyl, substituted C₁₋₆ alkylamino C₂₋₆ alkyl, di(substituted C₁₋₆ alkyl)amino C₂₋₆ alkyl, substituted C₁₋₆ alkylthio C₂₋₆ alkyl, in which the substituents are selected from the group consisting of pyrrolidino, piperidino morpholino, piperazino, 4-N—C₁₋₆ alkylpiperazino, 4-N—C₃₋₆alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆alkylamino C₁₋₆ alkyl)piperazino, 4-N—(C₁₋₆ alkylamino C₃₋₆ alkenyl)piperazino, imidazolyl, oxazolyl, thiazolyl,
(d) C₁₋₆CONR₆R₇, or trans-CH═CHCONR₆R₇, wherein R₆ and R₇ are independently selected from the group consisting of C₁₋₆ alkyl, phenyl C₁₋₆ alkyl, C₁₋₆ alkoxycarbonylmethyleneoxy, hydroxy C₂₋₆ alkyl, C₁₋₆ alkyloxy C₂₋₆ alkyl, amino C₂₋₆ alkyl, C₁₋₆ alkylamino C₂₋₆ alkyl, di(C₁₋₆ alkyl)amino C₂₋₆ alkyl, C₁₋₆ alkylthio C₂₋₆ alkyl, substituted C₁₋₆ alkoxy C₂₋₆ alkyl, substituted C₁₋₆ alkylamino C₂₋₆ alkyl, di(substituted C₁₋₆ alkyl)amino C₂₋₆ alkyl, substituted C₁₋₆ alkylthio C₂₋₆ alkyl, wherein the substituents are selected from the group consisting of pyrrolidino, piperidino, morpholino, piperazino, 4-N— C₁₋₆ alkylpiperazino, 4-N—C₃₋₆ alkenylpiperazino, 4-N—(C₁₋₆alkoxy C₁₋₆ alkyl)piperazino, 4-N—(C₁₋₆alkoxy C₃₋₆ alkenyl)piperazino, 4-N—(C₁₋₆alkylamino C₁₋₆ alkyl)piperazino, 4-N—(C₁₋₆alkylamino C₃₋₆ alkenyl)piperazino, imidazolyl, oxazolyl, thiazolyl,
(e) R₇ C(O) C₁₋₆ alkyl, R₇ C(O) C₂₋₆ alkenyl, in which R₇ is defined as above [2(d)],
(f) HO—C₁₋₆ alkyl-C₂₋₆ alkenyl, R₇—O—C₁₋₆ alkyl-C₂₋₆ alkenyl, R₇NH—C₁₋₆alkyl-C₂₋₆ alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆alkyl-C₂₋₆alkenyl, R₇O—C(O)—O—C₁₋₆alkyl-C₂₋₆alkenyl, R₇—C(O)—O—C₁₋₆ alkyl-C₂₋₆ alkenyl, wherein R₆ and R₇ is defined as above [2(d)],
(g) R₇—O—C₀₋₃ alkyl-C₃₋₆ cycloalkan-1-yl, R₇NH— C₀₋₃ alkyl- C₃₋₆ cycloalkan-1-yl, R₆R₇N—C₀₋₃ alkyl- C₃₋₆ cycloalkan-1-yl, R₇NH—C(O)—O— C₀₋₃ C₃₋₆ cycloalkan-1-yl, R₆R₇N—C(O)—O— C₀₋₃ alkyl- C₃₋₆ cycloalkan-1-yl, R₇O— C(O)—O— C₀₋₃ alkyl- C₃₋₆ cycloalkan-1-yl, R₇—C(O)—O— C₀₋₃ alkyl- C₃₋₆ cycloalkan-1-yl, R₇O—C(O)—C₀₋₃ alkyl- C₃₋₆ cycloalkan-1-yl, wherein R₇ and is defined as above [B(d)];

then R₂ and R₃ are each independently selected from the group consisting of:
(1) hydrogen, halo, trihalomethyl, C₁₋₆ alkyl, substituted C₁₋₆ alkyl, C₂₋₆ alkenyl, substituted C₁₋₆ alkenyl, C₁₋₆ alkyloxy, substituted C₁₋₆ alkyloxy, C₃₋₆ alkenyloxy, substituted C₃₋₆ alkenyloxy, C₁₋₆ alkylamino, substituted C₁₋₆ alkylamino, C₃₋₆ alkenylamino, substituted C₃₋₆ alkenylamino,
(2) mono-, di-, and tri-substituted phenyl wherein the substituents are independently selected from:
   (i) halo, trifluoromethyl, substituted C₁₋₆ alkyl,
   (ii) C₁₋₆ alkyloxy, substituted C₁₋₆ alkyloxy, C₃₋₆ alkenyloxy, substituted C₃₋₆ alkenyloxy,
   (iii) C₁₋₆ alkyl-amino, di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, di(substituted C₃₋₆ alkenyl)amino, or
   (iv) pyrrolidino, piperidino, morpholino, imidazolyl, substituted imidazolyl, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, 4-N—(C₁₋₆ alkylamino C₃₋₆ alkenyl)piperazino,
wherein the substituents are selected from the group consisting of:
   (a) hydrogen, hydroxy, halo, trifluoromethyl,
   (b) C₁₋₆ alkylalkoxy, C₁₋₆ alkylamino, C₁₋₆ alkylthio,
   (c) C₃₋₆ alkenyloxy, C₃₋₆ alkenylamino, C₃₋₆ alkenylthio, or
   (d) pyrrolidino, piperidino, morpholino, imidazolyl, substituted imidazolyl, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, 4-N—(C₁₋₆ alkylamino C₃₋₆ alkenyl)piperazino;

with the proviso that at least one of R₂ and R₃ group be selected from [B (2)] and the phenyl and the substituents be selected from (ii)-(v) above; or R₂ and R₃ taken together forming an aryl group such as phenyl, pyridyl, in which the aryl may be optionally substituted, wherein the substituents are defined as above in (i)-(iv);

and R₄ is selected from the group consisting of:
(a) hydrogen;
(b) substituted C₁₋₁₁ alkyl or C₂₋₁₁ alkenyl wherein the substituents are independently selected from the group consisting of:
   (i) hydrogen, hydroxy, C₁₋₆ alkyloxy, C₁₋₆alkylthio, C₁₋₆ alkylamino, phenyl-C₁₋₆ alkylamino, C₁₋₆ alkoxycarbonyl;
   (ii) substituted C₁₋₆ alkyloxy, C₃₋₆ alkenyloxy, substituted C₃₋₆ alkenyloxy,
   (iii) di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, di(substituted C₃₋₆ alkenyl)amino; and
   (iv) pyrrolidino, piperidino, morpholino, imidazolyl, substituted imidazolyl, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, and 4-N—(C₁₋₆ alkylamino C₃₋₆ alkenyl)piperazino; and
(b) aryl C₀₋₁₁ alkyl wherein the aryl group is selected from phenyl, imidazolyl, furyl, thienyl.

In some embodiments, the invention provides a compound of Formula 1a, in the form of a free compound or its pharmaceutically acceptable pro-drug, metabolite, analogue, derivative, solvate or salt, for use in the methods of the invention, wherein:

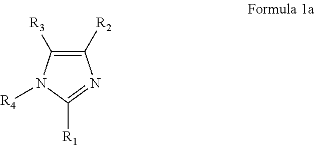

wherein the substituents R₁, R₂, R₃, and R₄ are defined as in A or B:
(B) R₁ is selected from the group consisting of:
(i) substituted C₁₋₁₁ alkyl or substituted C₂₋₁₁ alkenyl, wherein the substituents are selected from the group consisting of hydroxy and C₁₋₆ alkyloxy; and
(ii) mono-, di-, or tri-substituted aryl-C₀₋₁₁ alkyl wherein aryl is selected from the group consisting of phenyl, furyl, and thienyl wherein the substituents are selected from the group consisting of:
   (a) phenyl, trans-2-phenylethenyl, 2-phenylethynyl, or 2-phenylethyl, wherein the phenyl group is mono- or disubstituted wherein the substituents are selected from the group consisting of hydroxy, halo, C₁₋₄ alkyl and C₁₋₄ alkyloxy;
   (b) substituted C₁₋₆ alkyl, substituted C₂₋₆ alkyloxy, substituted C₂₋₆ alkylthio, or substituted C₂₋₆ alkoxycarbonyl, wherein the substituents are selected from the group consisting of C₁₋₆ alkoxy, and C₁₋₆ alkylthio; and
   (c) C₁₋₁₁ CO₂R₅, C₁₋₁₁CONHR₅, trans-CH═CHCO₂R₅, or trans-CH═CHCONHR₅ wherein R₅ is C₁₋₁₁ alkyl, phenyl C₁₋₁₁ alkyl, or C₁₋₆ alkoxycarbonylmethyleneoxy;

R₂ and R₃ are each independently selected from the group consisting of mono-, di, and tri-substituted phenyl wherein the substituents are independently selected from:
(i) substituted C₁₋₆ alkyl;
(ii) substituted C₁₋₆ alkyloxy, C₃₋₆ alkenyloxy, or substituted C₃₋₆ alkenyloxy;
(iii) substituted C₁₋₆ alkyl-amino, di(substituted C₁₋₆ alkyl)amino;
(iv) C₃₋₆ alkenyl-amino, di(C₃₋₆ alkenyl)amino, substituted C₃₋₆ alkenyl-amino, or di(substituted C₃₋₆ alkenyl)amino; and
(v) pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy-C₁₋₆ alkyl)piperazino, 4-N—(C₁₋₆ alkoxy-C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino;

wherein the substituents for (i), (ii), (iii), and (iv) are selected from the group consisting of:
(a) hydroxy, C₁₋₆ alkoxy, or C₁₋₆ alkylamino;
(b) C₃₋₆ alkenyloxy, or C₃₋₆ alkenylamino; and
(c) pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino;

or R₂ and R₃ are taken together to form an aryl group or substituted aryl, wherein the substituents are defined as above in (i)-(iv);

and R₄ is selected from the group consisting of:
(i) hydrogen;
(ii) substituted C₁₋₁₁ alkyl or C₂₋₁₁ alkenyl wherein the substituents are independently selected from the group consisting of hydrogen, hydroxy, C₁₋₆ alkyloxy, C₁₋₆alkylthio, C₁₋₆ alkylamino, phenyl-C₁₋₆ alkylamino, and C₁₋₆ alkoxycarbonyl; and
(iii) substituted aryl C₀₋₁₁ alkyl wherein the aryl group is selected from phenyl, imidazolyl, furyl, and thienyl in which the substituents are selected from the group consisting of:
   (a) hydroxy, C₁₋₆ alkoxy, or C₁₋₆ alkylamino;
   (b) C₃₋₆ alkenyloxy, or C₃₋₆ alkenylamino; and
   (c)pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino; or

(B) R₁ is selected from the group consisting of:
mono-, di-, and tri-substituted aryl-C₀₋₆ alkyl wherein aryl is selected from the group consisting of phenyl and thienyl, and the substituents are selected from the group consisting of:
(i) trans-2-substituted benzimidazolylethenyl, trans-2-substituted benzoxazolylethenyl, or trans-2-substituted benzthiazolylethenyl, in which the substituents are selected from the group consisting of hydrogen, hydroxy, halo, trihalomethyl, C₁₋₄ alkyl, C₁₋₄ alkyloxy, C₁₋₄ alkyloxycarbonyl, C₁₋₄ alkylamino, alkyl)amino, C₃₋₆ alkenylamino, di(C₃₋₆ alkenyl)amino, C₁₋₄ alkyloxy-C₁₄ alkylamino, substituted C₁₋₄ alkyl, substituted C₁₋₄ alkyloxy, substituted C₁₋₄ alkyloxycarbonyl, substituted C₁₋₄ alkylamino, di(substituted C₁₋₄ alkyl)amino, substituted C₃₋₆ alkenylamino, and di(substituted C₃₋₆ alkenyl)amino, wherein the substituents are selected from the group consisting of:
   (a) hydroxy, C₁₋₆ alkoxy, or C₁₋₆ alkylamino;
   (b) C₃₋₆ alkenyloxy, or C₃₋₆ alkenylamino; and
   (c)pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino;
(ii) trans-2-cyano ethenyl, trans-2-alkylsulfonyl ethenyl, trans-2-alkenylsulfonyl ethenyl, trans-2-substituted alkylsulfonyl ethenyl, and trans-2-substituted alkenylsulfonyl ethenyl, wherein the substituents are selected from the group consisting of:
   (a) hydroxy, C₁₋₆ alkoxy, or C₁₋₆ alkylamino;
   (b) C₃₋₆ alkenyloxy, or C₃₋₆ alkenylamino; and
   (c)pyrrolidino, piperidino, morpholino, imidazolyl, substituted imidazolyl, piperazino, 4-N—C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino-C₁₋₆ alkyl)piperazino, or 4-N—(C₁₋₆ alkylamino-C₃₋₆ alkenyl)piperazino;
(iii) C₁₋₆ CO₂R₅, trans-CH═CHCO₂R₅, C₁₋₆CONHR₅, or trans-CH═CHCONHR₅, wherein R₅ is C₁₋₆ alkoxy-C₂₋₆ alkyl, amino-C₂₋₆ alkyl, C₁₋₆ alkylamino-C₂₋₆ alkyl, di(C₁₋₆ alkyl)amino-C₂₋₆ alkyl, C₁₋₆ alkylthio-C₂₋₆ alkyl, substituted C₁₋₆ alkoxy-C₂₋₆ alkyl, substituted C₁₋₆ alkylamino-C₂₋₆ alkyl, di(substituted C₁₋₆ alkyl)amino-C₂₋₆ alkyl, or substituted C₁₋₆ alkylthio-C₂₋₆ alkyl, in which the substituents are selected from the group consisting of pyrrolidino, piperidino morpholino, piperazino, 4-N—C₁₋₆ alkylpiperazino, 4-N—C₃₋₆ alkenylpiperazino, 4-N—(C₁₋₆ alkoxy-C₁₋₆ alkyl)piperazino, 4-N—(C₁₋₆ alkoxy-C₃₋₆ alkenyl)piperazino, 4-N—(C₁₋₆ alkylamino-C₁₋₆ alkyl)piperazino, 4-N—(C₁₋₆ alkylamino C₃₋₆ alkenyl)piperazino, imidazolyl, oxazolyl, and thiazolyl;
(iv) C₁₋₆CONHR₅, or trans-CH═CHCONR₆R₇, wherein R₆ and R₇ are independently selected from the group consisting of C₁₋₆ alkyl, phenyl-C₁₋₆ alkyl, C₁₋₆ alkoxycarbonylmethyleneoxy, hydroxy-C₂₋₆ alkyl, C₁₋₆ alkyloxy-C₂₋₆ alkyl, amino-C₂₋₆ alkyl, C₁₋₆ alkylamino-C₂₋₆ alkyl, di(C₁₋₆ alkyl)amino-C₂₋₆ alkyl, C₁₋₆ alkylthio-C₂₋₆ alkyl, substituted C₁₋₆ alkoxy-C₂₋₆ alkyl, substituted C₁₋₆ alkylamino-C₂₋₆ alkyl, di(substituted C₁₋₆ alkyl)amino-C₂₋₆ alkyl, substituted C₁₋₆ alkylthio-C₂₋₆ alkyl, wherein the substituents are selected from the group consisting of pyrrolidino, piperidino, morpholino, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy-C₃₋₆ alkenyl)piperazino, 4-N—(C₁₋₆ alkylamino-C₁₋₆ alkyl)piperazino, 4-N—(C₁₋₆ alkylamino-C₃₋₆ alkenyl)piperazino, imidazolyl, oxazolyl, and thiazolyl;
(v) R₇—C(O) -C₁₋₆ alkyl or R₇—C(O) -C₂₋₆ alkenyl, in which R₇ is defined as above in [B(iv)];
(vi) HO—C₁₋₆ alkyl-C₂₋₆ alkenyl, R₇—O—C₁₋₆ alkyl-C₂₋₆ alkenyl, R₇NH—C₁₋₆alkyl-C₂₋₆ alkenyl, R₆R₇N—C₁₋₆alkyl-C₂₋₆alkenyl, R₇NH—C(O)—O—C₁₋₆alkyl-C₂₋₆alkenyl,

R₆R₇N—C(O)—O—C₁₋₆alkyl-C₂₋₆alkenyl, R₇O—C(O)—O—C₁₋₆alkyl-C₂₋₆alkenyl, or R₇—C(O)—O—C₁₋₆ alkyl-C₂₋₆ alkenyl, wherein R₆ and R₇ is defined as above in [B(iv)]; and
(vii) R₇—O—C₀₋₃ alkyl-C₃₋₆ cycloalk-1-yl, R₇NH— C₀₋₃ alkyl- C₃₋₆ cycloalk-1-yl, R₆R₇N— C₀₋₃ alkyl- C₃₋₆ cycloalk-1-yl, R₇NH—C(O)—O— C₀₋₃ C₃₋₆ cycloalk-1-yl, R₆R₇N—C(O)—O— C₀₋₃ alkyl- C₃₋₆ cycloalk-1-yl, R₇O— C(O)—O— C₀₋₃ alkyl- C₃₋₆ cycloalk-1-yl, R₇—C(O)—O—C₀₋₃ alkyl- C₃₋₆ cycloalk-1-yl, R₇O—C(O)—C₀₋₃ alkyl- C₃₋₆ cycloalk-1-yl, wherein R₇ and R₆ are defined as above in [B(iv)];

R₂ and R₃ are each independently selected from the group consisting of:
(viii) hydrogen, halo, trihalomethyl, C₁₋₆ alkyl, substituted C₁₋₆ alkyl, C₂₋₆ alkenyl, substituted C₂₋₆ alkenyl, C₁₋₆ alkyloxy, substituted C₁₋₆ alkyloxy, C₃₋₆ alkenyloxy, substituted C₃₋₆ alkenyloxy, C₁₋₆ alkylamino, substituted C₁₋₆ alkylamino, C₃₋₆ alkenylamino, or substituted C₃₋₆ alkenylamino; and
(ix) mono-, di-, or tri-substituted phenyl wherein the substituents are independently selected from the group consisting of:
(a) halo, trifluoromethyl, or substituted C₁₋₆ alkyl;
(b) C₁₋₆ alkyloxy, substituted C₁₋₆ alkyloxy, C₃₋₆ alkenyloxy, substituted C₃₋₆ alkenyloxy;
(c) C₁₋₆ alkyl-amino, di(C₁₋₆alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, or di(substituted C₃₋₆ alkenyl)amino; and
(d) pyrrolidino, piperidino, morpholino, imidazolyl, substituted imidazolyl, piperazino, 4-N— C₁₋₆ alkylpiperazino, 4-N—C₃₋₆alkenylpiperazino, 4-N—(C₁₋₆alkoxy C₁₋₆ alkyl)piperazino, 4-N—(C₁₋₆alkoxy C₃₋₆ alkenyl)piperazino, 4-N—(C₁₋₆alkylamino C₁₋₆ alkyl)piperazino, or 4-N—(C₁₋₆alkylamino C₃₋₆ alkenyl)piperazino;
   wherein the substituents for (a), (b), (c), and (d) are selected from the group consisting of:
(1) hydrogen, hydroxy, halo, or trifluoromethyl;
(2) C₁₋₆ alkylalkoxy, C₁₋₆ alkylamino, or C₁₋₆ alkylthio;
(3) C₃₋₆ alkenyloxy, C₃₋₆ alkenylamino, or C₃₋₆ alkenylthio; and
(4) pyrrolidino, piperidino, morpholino, imidazolyl, substituted imidazolyl, piperazino, 4-N— C₁₋₆ alkylpiperazino, 4-N—C₃₋₆alkenylpiperazino, 4-N—(C₁₋₆alkoxy C₁₋₆ alkyl)piperazino, 4-N—(C₁₋₆alkoxy C₃₋₆ alkenyl)piperazino, 4-N—(C₁₋₆alkylamino C₁₋₆ alkyl)piperazino, or 4-N—(C₁₋₆alkylamino C₃₋₆ alkenyl)piperazino;

with the proviso that a) at least one of R₂ and R₃ is selected from [B (ix)] and wherein the substituents are selected from [B (ix) (b)-(d)] above; or b) R₂ and R₃ are taken together to form an optionally substituted aryl group, wherein the substituents are defined as above in [B (ix) (a)-(d)];

and R₄ is selected from the group consisting of:
(i) hydrogen;
(ii) substituted C₁₋₁₁ alkyl or C₂₋₁₁ alkenyl wherein the substituents are independently selected from the group consisting of:
   (a) hydrogen, hydroxy, C₁₋₆ alkyloxy, C₁₋₆alkylthio, C₁₋₆ alkylamino, phenyl-C₁₋₆ alkylamino, or C₁₋₆ alkoxycarbonyl;
   (b) substituted C₁₋₆ alkyloxy, C₃₋₆ alkenyloxy, or substituted C₃₋₆ alkenyloxy;
   (c) di(C₁₋₆alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, or di(substituted C₃₋₆ alkenyl)amino; and
   (d) pyrrolidino, piperidino, morpholino, imidazolyl, substituted imidazolyl, piperazino, 4-N— C₁₋₆ alkylpiperazino, 4-N—C₃₋₆alkenylpiperazino, 4-N—(C₁₋₆alkoxy C₁₋₆ alkyl)piperazino, 4-N—(C₁₋₆ alkoxy C₃₋₆ alkenyl)piperazino, 4-N—(C₁₋₆alkylamino C₁₋₆ alkyl)piperazino, or 4-N—(C₁₋₆alkylamino C₃₋₆ alkenyl)piperazino; and
(iii) aryl C₀₋₁₁ alkyl wherein the aryl group is selected from phenyl, imidazolyl, furyl, or thienyl.

In some embodiments of the invention, the compound of Formula 1a is a compound wherein R₁ is selected from the group consisting of mono-, di-, and tri-substituted aryl-Cₘ alkyl wherein aryl is selected from the group consisting of phenyl and thienyl, and the substituents are selected from the group consisting of:
(a) C₁₋₆ CO₂R₅, trans-CH═CHCO₂R₅, C₁₋₆CONHR₅, or trans-CH═CHCONHR₅;
(b) C₁₋₆CONR₆R₇, or trans-CH═CHCONR₆R₇;
(c) R₇ C(O) C₁₋₆ alkyl or R₇ C(O) C₂₋₆ alkenyl; and
(d) HO—C₁₋₆ alkyl-C₂₋₆ alkenyl, R₇—O—C₁₋₆ alkyl-C₂₋₆ alkenyl, R₇NH—C₁₋₆alkyl-C₂₋₆ alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆alkyl-C₂₋₆alkenyl, R₇O—C(O)—O—C₁₋₆alkyl-C₂₋₆alkenyl, or R₇—C(O)—O—C₁₋₆ alkyl-C₂₋₆ alkenyl.

In other embodiments, the compound of Formula 1a is a compound wherein R₁ is selected from the group consisting of mono-, di-, and tri-substituted aryl-Cₘ alkyl wherein aryl is selected from the group consisting of phenyl and thienyl, and the substituents are selected from the group consisting of:
(a) C₁₋₆ CO₂R₅, trans-CH═CHCO₂R₅, C₁₋₆CONHR₅, or trans-CH═CHCONHR₅;
(b) C₁₋₆CONR₆R₇, or trans-CH═CHCONR₆R₇;
(c) R₇ C(O) C₁₋₆ alkyl or R₇ C(O) C₂₋₆ alkenyl; and
(d) HO—C₁₋₆ alkyl-C₂₋₆ alkenyl, R₇—O—C₁₋₆ alkyl-C₂₋₆ alkenyl, R₇NH—C₁₋₆alkyl-C₂₋₆ alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆alkyl-C₂₋₆alkenyl, R₇O—C(O)—O—C₁₋₆alkyl-C₂₋₆alkenyl, or R₇—C(O)—O—C₁₋₆ alkyl-C₂₋₆ alkenyl.

In various embodiments of the invention, the compound of Formula 1a is a compound wherein R₁ is selected from the group consisting of mono-, di-, and tri-substituted aryl-Cₘ alkyl wherein aryl is selected from the group consisting of phenyl and thienyl, and the substituents are HO—C₁₋₆ alkyl-C₂₋₆ alkenyl, R₇—O—C₁₋₆ alkyl-C₂₋₆ alkenyl, R₇NH—C₁₋₆ alkyl-C₂₋₆ alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆ alkyl-C₂₋₆ alkenyl, R₇O—C(O)—O—C₁₋₆ alkyl-C₂₋₆ alkenyl, or R₇—C(O)—O—C₁₋₆ alkyl-C₂₋₆ alkenyl.

In other embodiments, R₁ is selected from the group consisting of mono-, di-, and tri-substituted aryl-C₀₋₆ alkyl wherein the aryl-C₀₋₆ alkyl is phenyl-C₀₋₆ alkyl. In some embodiments, R₁ is selected from the group consisting of mono-, di-, and tri-substituted aryl-C₀₋₆ alkyl wherein the aryl-C₀₋₆ alkyl is aryl-C₀alkyl, which is aryl with no alkyl group attached directly to aryl.

In various embodiments, R₂ and R₃ are each independently selected from the group consisting of: mono-, di-, and tri-substituted phenyl wherein the substituents are independently selected from the group consisting of:
(i) C₁₋₆ alkyloxy, substituted C₁₋₆ alkyloxy, C₃₋₆ alkenyloxy, or substituted C₃₋₆ alkenyloxy;
(ii) C₁₋₆ alkyl-amino, di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, or di(substituted C₃₋₆ alkenyl)amino, and
(iv) pyrrolidino, piperidino, morpholino, imidazolyl, substituted imidazolyl, piperazino, 4-N— C₁₋₆ alkylpiperazino, 4-N—C₃₋₆ alkenylpiperazino, 4-N—(C₁₋₆ alkoxy C₁₋₆ alkyl)piperazino, 4-N—(C₁₋₆ alkoxy C₃₋₆ alkenyl)piperazino, 4-N—(C₁₋₆ alkylamino C₁₋₆ alkyl)piperazino, or 4-N—(C₁₋₆ alkylamino C₃₋₆ alkenyl)piperazino.

In some embodiments, R₂ and R₃ are each independently selected from the group consisting of: mono-, di-, and tri-substituted phenyl wherein the substituents are independently selected from the group consisting of C₁₋₆ alkyl-amino, di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, and di(substituted C₃₋₆ alkenyl)amino. In some embodiments, R₄ is hydrogen.

In some embodiments, the compound of Formula 1a is a compound of Formula 1b:

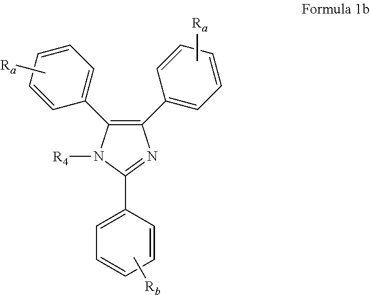

wherein each instance of Rₐ is independently C₁₋₆ alkyl-amino, di(C₁₋₆ alkyl)amino, substituted C₁₋₆ alkyl-amino, di(substituted C₁₋₆ alkyl)amino, C₃₋₆ alkenyl-amino, di(C₃₋₆ alkenyl)amino, substituted C₃₋₆ alkenyl-amino, or di(substituted C₃₋₆ alkenyl)amino; and

R_{b} is HO—C₁₋₆ alkyl-C₂₋₆ alkenyl, R₇—O—C₁₋₆ alkyl-C₂₋₆ alkenyl, R₇NH—C₁₋₆ alkyl-C₂₋₆ alkenyl, R₆R₇N—C₁₋₆ alkyl-C₂₋₆ alkenyl, R₇NH—C(O)—O—C₁₋₆ alkyl-C₂₋₆ alkenyl, R₆R₇N—C(O)—O—C₁₋₆ alkyl-C₂₋₆ alkenyl, R₇O—C(O)—O—C₁₋₆ alkyl-C₂₋₆ alkenyl, or R₇—C(O)—O—C₁₋₆ alkyl-C₂₋₆ alkenyl.

In some embodiments, the compound of Formula 1 or 1a (such as a compound of Formula 1b or 2), is in the form of a free compound or as its pharmaceutically-acceptable pro-drug, metabolite, analogue, derivative, solvate or salt, and is selected from the group consisting of: (2[4-(3-ethoxy-1-propenyl)phenyl]-4,5-bis(4-(2-propylamino)phenyl)-1H-imidazole; 2-[4-(3-ethoxy-trans-1-pro-pen-1-yl)phenyl]-4,5-bis(4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2[4-(3-methoxy-trans-1-propen-1-yl)ph-enyl]-4,5-bis(4-pyrrolidinophenyl) imidazole; 2-[4-(3-ethoxy-trans-1-prop-en-1-yl)phenyl]-4,5-bis(4-pyrrolidinophenyl) imidazole; 2-[4-(3-ethoxy-trans-1-propen-1-yl)phenyl]-4-(4-N-dimethylaminophenyl)-5 (4 pyrrolidinophenyl) imidazole; 2[4-(3-ethoxy-trans-1-propen-1-yl)phenyl-]-4-(4-N methylaminophenyl)-5-(4-pyrrolidino-phenyl) imidazole; 2-[4-(3-ethoxy-trans-1-propen-1-yl)phenyl]-4,5-bis(4-N-morpholinophenyl) imidazole; 2-[4-(3-ethoxy-trans-1-propen-1-yl)phenyl]-4-(4-N-dimethylamin-ophenyl)-5-(4-N-morpholinophenyl) imidazole; 2-[4-(3-ethoxy-trans-1-propen 1 yl)phenyl]-4-(4-N-methylaminophenyl)-5-(4-N-morpholinophenyl) imidazole; and 2[4-(3-ethoxy-trans-1-propen-1-yl)phenyl]-4-4-N-methylami-nophenyl)-5-(4-N-isopropylaminophenyl) imidazole.

The compound of Formula 1 or 1a can be the specific formulas as described in U.S. Pat. Nos. 5,700,826 and 5,840,721, herein incorporated by reference. Preferred compositions and methods comprise the compound of the following formula (Formula 2):

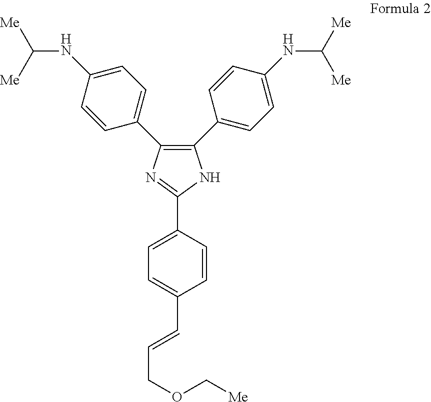 [SMILES: CCOC/C=C/c1ccc(-c2nc(-c3ccc(NC(C)C)cc3)c(-c3ccc(NC(C)C)cc3)[nH]2)cc1]

in the form of a free compound or as its pharmaceutically-acceptable pro-drug, metabolite, analogue, derivative, solvate or salt.

The compounds of Formula 1 or 1a (such as a compound of Formula 1b or 2) are synthesized by any suitable method known in the field. Examples of the synthesis for this class of compounds and the compound of Formula 2, in particular, are disclosed in U.S. Pat. No. 5,840,721, which is hereby incorporated by reference in its entirety.

ii. MDR Inhibitors

The compounds of the present invention modulate GSL synthesis and/or metabolism and modulate α-synuclein function. The compounds can prevent accumulation of complex GSLs. The compounds can inhibit longer chain GSL formation, or complex GSL formation. The compounds can modulate GSL synthesis and/or metabolism by modulating the activity of an ABC transporter involved in GSL biosynthesis. The ABC transporter can be the P-glycoprotein, encoded by the MDR1 gene. MDR1 encodes a 170 kDa membrane glycoprotein (gp-170 or Pgp) that typically acts as an ATP-dependent efflux pump, transporting a number of unrelated organic compounds out of the cell (Juranka et al., FASEB J. 3:2583-2592 (1989)). The level of expression of gp-170 has been shown to correlate with the degree of drug resistance (Raderer and Sscheitharer, Cancer 72: 3553-3563 (1993)). Gp-170 appears to act as a pump that actively extrudes a wide variety of structurally unrelated compounds, including a full range of antineoplastic drugs. Another ATP-dependent membrane efflux pump, the product of the MRP gene, has also been implicated in the MDR phenomenon (Krishnamachary and Center, Cancer Res. 53:3658-3661 (1993)), as have other ATP-dependent and enzymatic mechanisms.

In certain embodiments compounds that modulate MDR can modulate GSL synthesis and include but are not limited to vinblastine, vincristine, etoposide, teniposide, doxorubicin (adriamycin), daunorubicin, pliamycin (mithramycin), and actinomycin D (Jones et al., Cancer (Suppl) 72:3484-3488 (1993)). Many tumors are intrinsically multidrug resistant (e.g., adenocarcinomas of the colon and kidney) while other tumors acquire MDR during the course of therapy (e.g., neuroblastomas and childhood leukemias). Recently, it has been shown that MDR cells, as opposed to drug-sensitive cells, display increased levels of glucosylceramide (Lavie et al., J. Biol. Chem 271:19530-19536271:19530-19536 (1996)) and further MDR modulators may increase the cellular susceptibility to chemotherapeutic agents through regulation of ceramide metabolism in cancer cells (Lavie et al., J. Biol. Chem 272:1682-1687 (1997)). Accumulation of glucosylceramide (GlcCer), a simple glycosylated form of ceramide, is a characteristic of some MDR cancer cells and tumors derived from patients who are less responsive to chemotherapy (Lavie .et al., J. Biol. Chem. 271:19530-19536 (1996); Lucc .et al., Anticancer Res. 18: 475-480 (1998)). Modification of ceramide metabolism, by blocking the glycosylation pathway, has been shown to increase cancer cell sensitivity to cytotoxics (Lucci et al., Int. J. Onc. 15: 541-546 (1999); Lavie et al., J. Biol. Chem. 272:1682-1687 (1997); Lucci et al., Cancer 86:299-310 (1999)). Further, drug combinations that enhance ceramide generation and limit glycosylation have been shown to enhance kill in cancer cell models (Lavie et al., J. Biol. Chem. 272:1682-1687 (1997); Lucci et al., Cancer 86:299-310 (1999)). Other work has shown that ceramide toxicity can be potentiated in experimental metastasis of murine Lewis lung carcinoma and human neuroepithelioma cells by inclusion of a glucosylceramide synthase inhibitor (Inokuchi et al., Cancer Res. 50: 6731-6737 (1990); Spinedi et al., Cell Death Differ. 5:785-791 (1998)).

In certain embodiments compounds described herein can modulate GSL levels by effecting MDR1 activity. The compounds can provide increased specificity for modulating GlcCer levels, as compared to modulating MDR. For example, a variety of structurally diverse agents have been identified which can restore partly or sometimes completely the normal drug sensitivity to some MDR tumor cells. These chemosensitizers are effective as a result of their ability to interfere with gp-170, causing a reversal in the increase in drug efflux, but among these agents are calcium channel blockers (e.g., verapamil), calmodulin inhibitors (e.g., trifluoperazine), antibiotica (e.g., erythromycin), cardiovascular agents (e.g., quinidine), noncytotoxic analogs of anthracyclines and vinca alkaloids, cyclosporin A and analogs thereof, FK-506 and analogs thereof, and derivatives of cyclopeptides (Lum et al., Cancer (Suppl) 72:3502-3514 (1993)). Many of these agents have not provided a significant contribution to the chemotherapeutic index for the treatment of cancer due to their significant pharmacological effects on other organ systems. Compounds of the present invention may be specific for the translocation or flippase activity of the MDR1 that affects GSL synthesis, rather than the reversal of MDR, and may also have a lack of significant toxicity and other nonspecific pharmacological effects. Alternatively, compounds may affect both, but have a greater effect on GSL levels rather than MDR.

For example, cells exhibiting abnormal GSL metabolism can be treated with the compounds of the present invention at a concentration or dosage that modulates GlcCer levels, but would not affect MDR in cancer cells. The compound administered to subjects suffering from GSL metabolism disorders can ameliorate symptoms of GSL disorder, but not MDR of subjects suffering from cancer. Therapeutically effective dosages of the compounds of the present invention can have an effect on GSL disorder symptoms, but not on MDR. In some embodiments, the compounds may specifically modulate the levels of specific GSL, for example neutral GSLs or acidic GSLs, or both, in which other MDR inhibitors do not. The compounds can have a higher specificity or increased activity in affecting GSL as compared to other MDR inhibitors, and thus more effective in treating GSL metabolism disorders. Dosages and toxicities can also vary of compounds that are used for treating GSL disorders as compared to treating MDR with MDR1 inhibitors.

Combinations of compounds of the present invention are also provided. In preferred embodiments, combinations have a synergistic effect. This invention contemplates administering the compounds with any of several different kinds of compounds. These include, for example, modulators of α-synuclein function, substrate competitors for enzyme inhibition therapy, enzymes for enzyme replacement therapy, gene therapy and chaperones for enzymes. For example, a composition of the present invention can comprise a first compound of Formula 1 as described herein, with a second compound that is a glucosyl ceramide synthase inhibitor. In some embodiments, the glucosyl ceramide synthase inhibitor is miglustat, or 1-butyl-2-(hydroxymethyl)piperidine-3,4,5-triol. Another compound that can be used is PDMP (1R-phenyl-2R-decanoylamino-3-morpholino-1-propanol), previously identified as the D-threo isomer (Inokuchi et al., J. Lipid Res. 28:565-571 (1987)), PDMP has been found to produce a variety of chemical and physiological changes in cells and animals (Radin et al., “Use of 1-Phenyl-2-Decanoylamino-3-Morpholino-1-Propanol (PDMP), an Inhibitor of Glucosylceramide Synthesis,” In NeuroProtocols, A Companion to Methods in Neurosciences, S. K. Fisher et al., Ed., (Academic Press, San Diego) 3:145-155 (1993) and Radin et al., “Metabolic Effects of Inhibiting Glucosylceramide Synthesis with PDMP and Other Substances,” In Advances in Lipid Research; Sphingolipids in Signaling, Part B., R. M. Bell et al., Ed. (Academic Press, San Diego) 28:183-213 (1993)). Homologs, analogs, or derivatives of PDMP can also be used, such as the P4 compound (1-phenyl-2-palmitoylamino-3-pyrrolidino-1-propanol). (Shayman et al., J. Biol. Chem., 277:18447-18453 (2002); Asano, Glycobiology 13:93R-104R (2003); Jimbo et al., J. Biochem. (Tokyo) 127:485-491 (2000)) Imino sugar-based glucosyl ceramide synthase inhibitors, such as N-butyldeoxynojirimycin, may also be used.

iii. Enzyme Replacement Therapy

In some embodiments, modulation of GSL comprises administering compositions comprising the compound of the present invention along with enzyme-replacement therapy (ERT), such as glucocerebrosidases or compounds that modulate glucocerebrosidases, for example with imiglucerase (an analogue of human β-glucocerebrosidase) or α-galactosidase (Brady, Acta Paediatr. Suppl. 92:19-24 (2003); Heukamp et al., Pathol. Res. Pract. 199:159-163 (2003); Wilcox et al., Am. J. Hum. Genet. 75:(65-74) (2004)). Combinatorial treatments also include gene therapy, for example, a patient with Fabry disease can be treated with a recombinant retrovirus carrying the cDNA encoding the defective α-Gal A that is used to transfect skin fibroblasts obtained from the Fabry patient (Medin et al., Proc. Natl. Acad. Sci. USA 93:7917-7922 (1996)) along with the compound of the present invention.

In another embodiment, the compound of Formula 1 is administered in combination with a chaperone. Chaperones have an important role in protein folding. Misfolded proteins are typically eliminated by cellular quality control mechanisms, or accumulate and affect protein trafficking. Artificial chaperones used in combination with the compound of the present invention include non-specific chemical chaperones, such as high concentrations of glycerol, dimethylsulfoxide, trimethylamine N-oxide, or deuterated water have been shown to stabilize the mutant protein and increase the intracellular trafficking of mutant protein in several diseases (Brown et al., Cell Stress Chaperones 1:117-125 (1996); Burrows et al., Proc. Natl. Acad. Sci. USA; 97: 1796-1801 (2000)). Pharmacological chaperones which bind to the enzyme and promote trafficking of the enzyme from the endoplasmic reticulum to the lysosome can be used. In preferred embodiments, the compound of Formula 1 is administered with active site specific chaperones (ASSC). ASSCs known in the art, such as 1-deoxygalactonojirimycin (DGJ) (U.S. Pat. Nos. 6,274,597, and 6,774,135), can be used. ASSCs are thought to stabilize misfolded proteins and enable proper protein conformation for trafficking to the lysosomes, and thus ASSCs aid in ameliorating LSDs (U.S. Pat. Nos. 6,583,158, 6,589,964, 6,599,919). Other ASSCs include glucoimidazole (GIZ) and polyhydroxycyclohexenyl amine (PHCA) derivatives (U.S. Patent Pub. No. 20050137223), which may be used in combination with the compound of the present invention for treating diseases associated with mutant glucocerebrosidase, such as Gaucher's. Hydroxy piperidine (HP) derivatives (U.S. Pat. Appin. 20050130972) can also be used in combination with the compound of Formula 1, for example, in treating individuals having Gaucher disease.

b. Other Agents Useful for Altering Lipid Metabolism

In certain embodiments other agents can be used to alter limit metabolism. In specific embodiments HMG Co A reductase inhibitors or statins can be used to alter lipid metabolism. In related embodiments agents that modulate cholesterol synthesis or fatty acid synthesis can be utilized to alter lipid metabolism. Such agents can be synthetic or naturally-derived. Exemplary statins include but are not limited to atorvastatin, cerivastatin, fluvastatin, lovastatin, mevastatin, pitavastatin, pravastatin, rosuvastatin, and simvastatin.

V. Methods of Diagnosis

(a) Parkinson's Disease and Related Diseases

In terms of diagnosis for Parkinson's disease (PD), there is no specific test or marker for PD. Typically, the diagnosis is based on medical history and neurological examination conducted by interviewing and observing the patient in person, which may include using the Unified Parkinson's Disease Rating Scale. A radiotracer for SPECT scanning machines called DaTSCAN is specialized for diagnosing dopamine loss characteristic of Parkinson's disease. The disease can be difficult to diagnose accurately, especially in its early stages due to symptom overlap with other causes of Parkinsonism. In some embodiments a premotor diagnosis is made. In other embodiments a genetic test is utilized. Physicians may need to observe the person for some time until it is apparent that the symptoms are consistently present. CT and MRI brain scans of people with PD are normal and therefore, not useful for diagnosis. However, doctors may sometimes request brain scans or laboratory tests in order to evaluate for other diseases that may produce signs of Parkinsonism.

To diagnose PD, the physician will perform a standard neurological examination, involving various simple tests of reactions, reflexes, and movements. Diagnosis of PD generally depends on the presence of at least two of the three major signs: tremor at rest, rigidity, and bradykinesia, as well as the absence of a secondary cause, such as antipsychotic medications or multiple small strokes in the regions of the brain controlling movement. Patients tend to be most aware of tremor and bradykinesia, and less so of rigidity. Bradykinesia is tested by determining how quickly the person can tap the finger and thumb together, or tap the foot up and down. Tremor is determined by simple inspection. The physician assesses rigidity by moving the neck, upper limbs, and lower limbs while the patient relaxes, feeling for resistance to movement. Postural instability is tested with the “pull test,” in which the examiner stands behind the patient and asks the patient to maintain their balance when pulled backwards. The examiner pulls back briskly to assess the patient's ability to recover, being careful to prevent the patient from falling. The examination also involves recording a careful medical history, especially for exposure to medications that can block dopamine function in the brain.

In other embodiments other physiological markers such as EKG, EEG, sleep behavior, are measured to diagnose PD, either prior to or following the onset of symptoms.

In some embodiments, the subjects that can be treated with the methods of the present invention are patients who experience one or more of the symptoms including but not limited to tremor of hands, arms, legs, jaw and face, stiffness or rigidity of the arms, legs and trunk, slowness of movement, poor balance and coordination, and postural instability. In some embodiments, the subjects that can be treated with the methods of the present invention are patients who have been diagnosed with Parkinson's disease by a physician. In some embodiments, the subjects that can be treated with the methods of the present invention are patients who have not been diagnosed with Parkinson's disease but are experiencing symptoms of PD.

(b) Gaucher Disease and Related Diseases

In terms of diagnosis for Gaucher disease or other related lipid storage disease, there may be no specific single test or marker for diagnosis. Typically, a diagnosis is based on medical history and examination conducted by interviewing and observing the patient in person, in conjunction with laboratory tests and other physiological variables. In the specific case of Gaucher disease, a definitive diagnosis is made with genetic testing. As there are numerous different mutations, sequencing of the beta-glucosidase gene is sometimes necessary to confirm the diagnosis. Prenatal diagnosis is available, and is useful when there is a known genetic risk factor.

A diagnosis can also be implied by biochemical abnormalities such as high alkaline phosphatase, angiotensin-converting enzyme (ACE) and immunoglobulin levels, or by cell analysis showing “crinkled paper” cytoplasm and glycolipid-laden macrophages.

VI. Methods of Use

A “patient,” “subject” or “host” to be treated with the composition of the present invention may mean either a human or non-human animal. The compounds of the present invention are useful in the treatment of diseases and disorders such as but not limited to neurological and lipid storage diseases. In one embodiment, the compositions of the present invention are used in the manufacture of a medicament for any number of uses, including for example treating neurological diseases and disorders, lysosomal storage diseases and disorders, or lipid metabolism diseases or disorders.

A “therapeutic effect,” as that term is used herein, encompasses a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. A prophylactic effect includes delaying or eliminating the appearance of a disease or condition, delaying or eliminating the onset of symptoms of a disease or condition, slowing, halting, or reversing the progression of a disease or condition, or any combination thereof.

The present invention also has the objective of providing suitable topical, oral, and parenteral pharmaceutical formulations for use in the novel methods of treatment of the present invention. The compounds of the present invention may be administered orally as tablets, aqueous or oily suspensions, lozenges, troches, powders, granules, emulsions, capsules, syrups or elixirs. The composition for oral use may contain one or more agents selected from the group of sweetening agents, flavoring agents, coloring agents and preserving agents in order to produce pharmaceutically palatable preparations. The tablets contain the acting ingredient in admixture with non-toxic pharmaceutically acceptable excipients that are suitable for the manufacture of tablets. These excipients may be, for example, (1) inert diluents, such as calcium carbonate, lactose, calcium phosphate, carboxymethylcellulose, or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in the U.S. Pat. Nos. 4,256,108; 4,160,452; and U.S. Pat. No. 4,265,874 to form osmotic therapeutic tablets for controlled release.

An effective amount of an agent of the current invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, aqueous, alcoholic, alcoholic-aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, growth factors and inert gases and the like.

Therefore, the present invention encompasses methods for ameliorating diseases and conditions, including but not limited to disorders associated with α-synuclein dysfunction and altered lipid metabolism with any of the α-synuclein modulating compounds, or lipid metabolism modulating compounds in the form of a free compound or a pharmaceutically-acceptable pro-drug, metabolite, analogue, derivative, solvate or salt, and a chemotherapeutic or pharmaceutical agent in an amount sufficient to inhibit or ameliorate the cell's proliferation or the disorder. Generally, the terms “treating”, “treatment” and the like are used herein to mean affecting a subject, tissue or cell to obtain a desired pharmacologic and/or physiologic effect. The effect may be prophylactic in terms of completely or partially preventing a disease or sign or symptom thereof, and/or may be therapeutic in terms of a partial or complete cure for a disorder and/or adverse effect attributable to, for example, aberrant cell proliferation. “Treating” as used herein covers any treatment of, or prevention of a disease or disorder in a vertebrate, a mammal, particularly a human, and includes: (a) preventing the disease or disorder from occurring in a subject that may be predisposed to the disease or disorder, but has not yet been diagnosed as having it; (b) inhibiting the disease or disorder, i.e., arresting its development; or (c) relieving or ameliorating the disease or disorder, i.e., cause regression of the disease or disorder.

The invention includes various pharmaceutical compositions useful for ameliorating diseases and disorders related to α-synuclein and lipid related disorders. The pharmaceutical compositions according to one embodiment of the invention are prepared using any of the compounds named herein in the form of a free compound or a pharmaceutically-acceptable pro-drug, metabolite, analogue, derivative, solvate or salt, and optionally, one or more pharmaceutical agents or combinations of the compounds into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions are preferably prepared and administered in dose units. Solid dose units are tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249:1527, (1990); Gilman et al. (eds.) (1990), each of which is herein incorporated by reference. Dosages for parenteral administration of active pharmaceutical agents can be converted into corresponding dosages for oral administration by multiplying parenteral dosages by appropriate conversion factors. As to general applications, the parenteral dosage in mg/m² times 1.8 may equal the corresponding oral dosage in milligrams (“mg”). See the Miller-Keane Encyclopedia & Dictionary of Medicine, Nursing & Allied Health, 5.sup.th Ed., (W.B. Saunders Co. 1992). pp. 1708 and 1651.

The method by which the compounds disclosed herein are administered for oral use would be, for example, in a hard gelatin capsule wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil. The active ingredient can be mixed with a co-solvent mixture, such as PEG 400 containing Tween-20. A compound can also be administered in the form of a sterile injectable aqueous or oleaginous solution or suspension. The compounds can generally be administered intravenously or as an oral dose of 0.5 to 10 mg/kg given every 12 hours, 1 to 3 times a day, or may be given before and 1 to 3 times after the administration of another pharmaceutical agent, with at least one dose 1 to 4 hours before and at least one dose within 8 to 12 hours after the administration of the other agent.

Aqueous suspensions normally contain the active materials in admixture with excipients suitable for the manufacture of aqueous suspension. Such excipients may be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleagenous suspension. This suspension may be formulated according to known methods using those suitable dispersing or wetting agents and suspending agents that have been mentioned above. The sterile injectable preparation can be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

A compound disclosed herein can also be administered in the form of suppositories for rectal administration of the drug. These compositions can be prepared by mixing the drug with a suitable non-irritating excipient that is solid at ordinary temperature but liquid at the rectal temperature and will therefore melt in the rectum to release the drug. Such materials include cocoa butter and polyethylene glycols.

The compounds as used in the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

For topical use, creams, ointments, jellies, solutions or suspensions, etc., containing the compounds disclosed herein may be employed.

Dosage levels of the compounds disclosed herein as used in the present invention may be of the order of about 0.5 mg to about 20 mg per kilogram body weight, an average adult weighing 70 kilograms, with a preferred dosage range between about 5 mg to about 20 mg per kilogram body weight per day (from about 0.3 gms to about 1.2 gms per patient per day). The amount of the compound that may be combined with the carrier materials to produce a single dosage will vary depending upon the host treated and the particular mode of administration. For example, a formulation intended for oral administration to humans may contain about 5 mg to 1 g of a compound disclosed herein with an appropriate and convenient amount of carrier material that may vary from about 5 to 95 percent of the total composition. Dosage unit forms will generally contain between from about 5 mg to 500 mg of the compound's active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the instant invention may form solvates with water or common organic solvents. Such solvates are encompassed within the scope of the invention.

In further embodiments the invention provides compositions comprising a compound disclosed herein in the form of pharmaceutically-acceptable pro-drugs, metabolites, analogues, derivatives, solvates or salts in admixture with an active pharmaceutical agent or chemotherapeutic agent, together with a pharmaceutically acceptable diluent, adjuvant, or carrier.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Materials and Methods

Conduritol B Epoxide Treatment In Vitro:

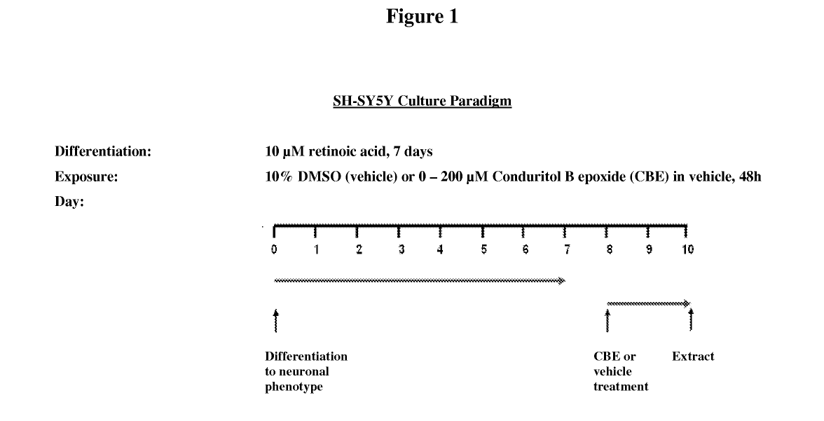

The treatment paradigm is depicted in FIG. 1. SH-SY5Y cells were grown in Dulbecco's modified Eagle medium with 10% fetal calf serum, 2 mM glutamine, and were subcultured 1:5 with TrypLE (GIBCO/Invitrogen; Carlsbad, Calif.) using standard tissue culture techniques. The cells were differentiated in neurobasal media supplemented with B-27 and 40 μM retinoic acid for 7 days (Pahlman et al., 1984). Cells were exposed to CBE at doses of 0, 12.5, 25, 50, 100 or 200 μM in dimethyl sulfoxide (DMSO; Sigma Chemicals; St. Louis, Mo.) for 48 h at 37° C. At this time, cultures were washed with HBSS and trypsinized for 10 min followed by centrifugation at 1,000×g for 10 min to pellet cells. Media was removed, and cells were lysed in 10 mM Tris/1 mM EDTA/protease inhibitor cocktail (1:1000; Sigma) by sonication. Samples were centrifuged at 1,000×g for 10 min; the supernatant fraction was decanted from particulate fraction. Following determination of protein concentration using the BCA protein assay (Pierce Chemicals, Rockford, Ill.), samples were frozen until used for Western blot analysis experiments.

Conduritol B Epoxide Administration In Vivo:

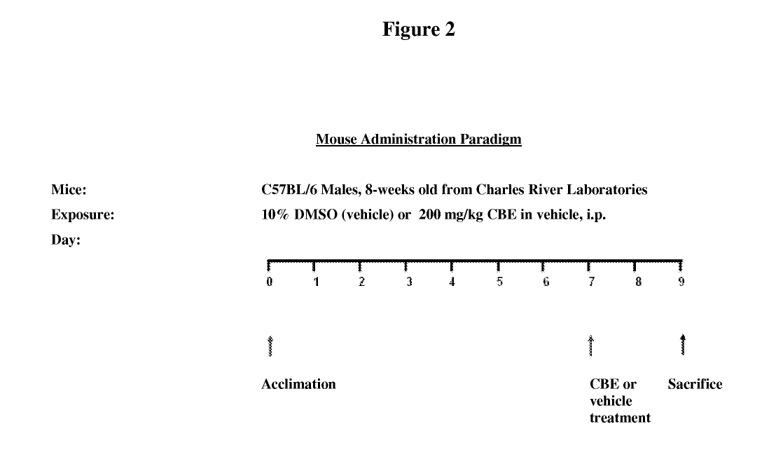

The administration paradigm is depicted in FIG. 2. Mice (C57BL/6) were maintained on a 12 h light-dark cycle and given food and drinking water ad libitum. All animal procedures and care methods were approved by the Institutional Animal Care and Usage Committee. In experiments to test the effects of CBE on α-synuclein protein, C57BL/6 male mice, aged 8 weeks, were used as previously described with some modifications (Kanfer et al., 1975, Adachi and Volk, 1977, Kanfer et al., 1982). Mice (n=8/group) received a single i. p. injection of 200 mg/kg CBE (Sigma) in DMSO or the vehicle alone and killed 2 days after injection. For Western blot analyses, brains were removed, dissected on ice and frozen on dry ice until needed (Manning-Bog et al., 2002, Purisai et al., 2005). For experiments utilizing immunohistochemistry, brains were immersion fixed in 4% paraformaldehyde and successively cryoprotected in 10 and 30% sucrose over the course of 72 h (Manning-Bog et al., 2002, Manning-Bog et al., 2003). Brains were sectioned at 40-μm intervals and stored in cryopreservative solution at −20° C. until needed.

Histochemistry:

Midbrain sections were immunostained using antibodies against α-synuclein (Syn-1; Transduction Laboratories; Lexington, Ky.) or glial acidic fibrillary protein (GFAP; Chemicon; Temecula, Calif.). Sections were then incubated with a FITC-conjugated species-specific secondary antibody and mounted onto slides as previously described (Manning-Bog et al. 2003).

RT-PCR:

RNA was extracted from human neuroblastoma cells (SH-SY5Y), treated for 24 h with CBE at varying doses or vehicle, using RAN Stat 60 (Testest; Friendswood, Tex.) according to manufacturer's instructions. The cDNAs were prepared by reverse transcription (Superscript III; Invitrogen). PCR was performed using the ABI PRISM 7000 Sequence Detection System and primers. The cycle number at which each PCR reaction reached a significant threshold (C_{T}) during the log phase of the amplification was used as a relative measure of transcript expression. The C_{T} of the α-synuclein gene was calibrated against that of the reference gene mouse HPRT.

Immunoblotting:

Fractions from ventral mesencephalon separated by the centrifugation were utilized for immunoblotting experiments. Following homogenization in 10 mM Tris/1 mM EDTA/protease inhibitor cocktail (1:1000; Sigma, St. Louis, Mo.) by sonication, samples were centrifuged at 1,000×g for 10 mM. The supernatant fraction (S1) was decanted and stored, and the pellet fraction containing nuclei and large membrane fragments were reconstituted in homogenization buffer (P1 fraction). The protein concentration was measured. After proteins were separated by SDS-PAGE and transferred to nitrocellulose, the blots were blocked and incubated overnight at 4° C. with anti-α-synuclein (Signet; Novus Biologicals, Littleton, Colo.; Abcam, Cambridge, Mass.; Santa Cruz Biotechnology, Santa Cruz, Calif.) or anti-GAPDH (Sigma). Appropriate secondary antibodies conjugated to HRP were applied, and blots were incubated with a chemiluminescent substrate (Pierce) and exposed to Kodak X-Omat Blue Film (Kodak, Rochester, N.Y.). Mouse or rabbit IgG was used in lieu of the primary antibody to ensure specificity in control experiments.

### Example 2

### In Vitro Effects of CBE Exposure

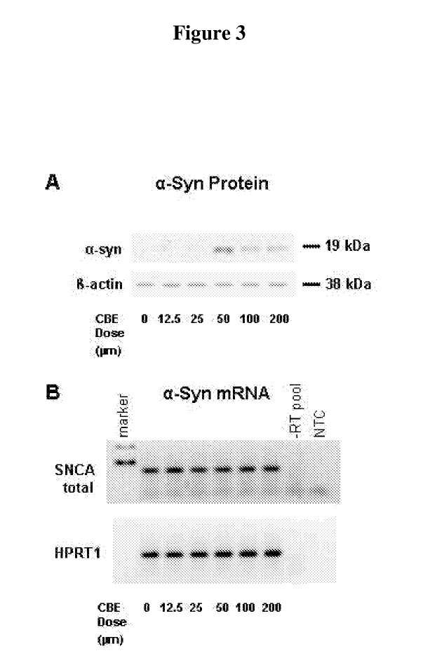

To test determine if the inhibition of GCase would elicit changes in cellular α-synuclein level, the protein levels were evaluated by using Western blot analysis in non-differentiated SH-SY5Y cells and cells differentiated to the neuronal phenotype, at 48 h following exposure to CBE. No change in α-synuclein was detected in non-differentiated neuroblastoma cells; however, in differentiated SH-SY5Y cells, increased levels of α-synuclein protein were observed, peaking at the 50 μM dose (FIG. 3a). In order to determine whether increased levels of the protein were due to enhanced transcription, RT-PCR was performed to measure transcript levels in SH-SY5Y treated with CBE. No change in α-synuclein gene expression was detected at any dose of the inhibitor tested at 24 h following CBE treatment. These findings indicate that increased α-synuclein levels observed following CBE exposure are not due to enhanced expression (FIG. 3b). It was noted there was no overt toxicity within cells treated with GCase inhibitor. In tissue culture, increased immunoreactivity for α-synuclein within CBE-exposed neuroblastoma cells differentiated to the neuronal phenotype was observed.

### Example 3

### In Vivo Effects of CBE Exposure

Protein Levels of α-Synuclein in the Substantia Nigra:

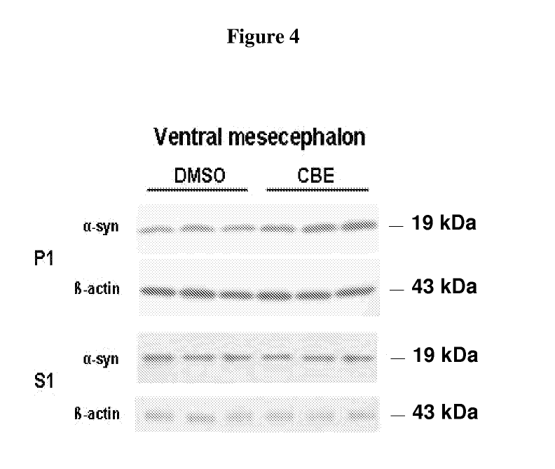

C57BL/6 mice were exposed to a single injection of CBE and assessed for changes in α-synuclein at 48 h to determine whether diminished GCase activity is associated with alterations in the protein in vivo, specifically within the substantia nigra. This schedule was chosen as previous reports have revealed enhanced α-synuclein levels at this time point (Vila et al., 2000, Manning-Bog et al., 2002). In tissue homogenates from ventral mesencephalon of CBE vs. DMSO (vehicle)-treated mice, α-synuclein immunoreactivity was assessed by Western blot analysis. Denser α-synuclein-positive bands, representing the monomeric form of the protein (at 19 kDa), were detected in the particulate fraction at 48 h following exposure to CBE vs. DMSO, with no alteration in the supernatant fraction (FIG. 4). No immunoreactivity for higher molecular forms of α-synuclein (i.e. SDS-stable aggregates) was observed under these conditions.

Protein Levels of α-Synuclein in the Ventral Mesencephalon:

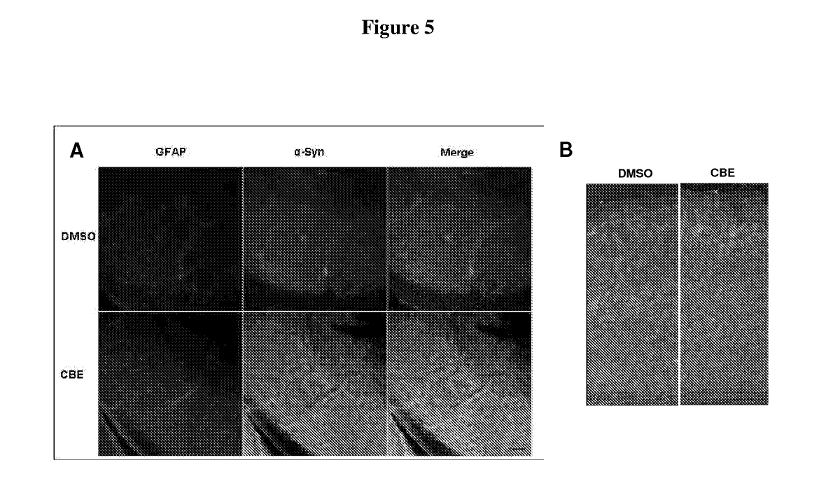

The effects of CBE exposure on α-synuclein within the ventral mesencephalon were also assessed histologically with immunohistochemistry. Coronal sections containing substantia nigra from mice at 48 h after CBE or DMSO exposure were immunostained using an antibody derived against α-synuclein (i.e. Syn-1). Subsequent evaluation of the sections revealed that robust immunoreactivity was observed within the cell bodies of the substantia nigra pars compacta of treated vs. control mice (FIG. 5a, 6), and enhanced immunoreactivity for α-synuclein was detected within the cytoplasm and cell nuclei (FIG. 5a, 6) of A9 neurons, reminiscent of the α-synuclein response in PD models of toxicant exposure (Vila et al. 2000; Manning-Bog et al. 2002; Goers et al. 2004). No obvious changes in α-synuclein were observed in other brain regions, such as the cortex (FIG. 5b) and hippocampus, 48 h following a single administration of CBE to mice.

Protein Levels in α-Synuclein in Astrocytes:

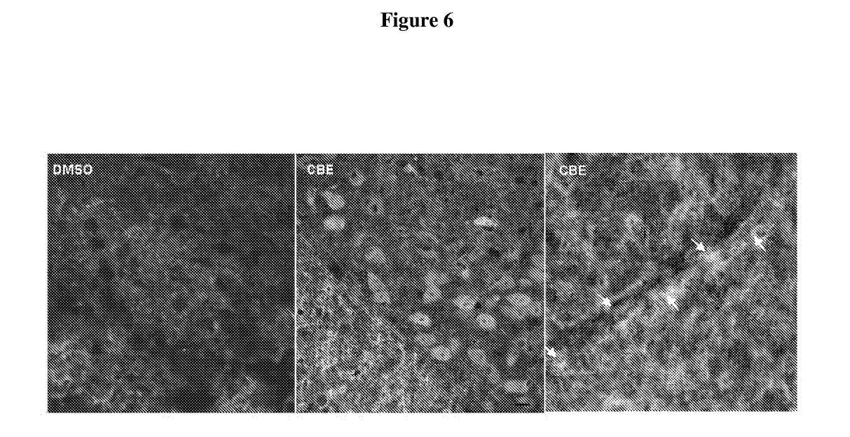

Substantia nigra-containing tissue sections were immunostained using an antibody for the astrocytic marker, glial fibrillary acidic protein (GFAP). At 48 h after exposure to a single systemic treatment, astroglial activation, as observed by GFAP immunoreactivity, was apparent in the substantia nigra (FIG. 6). Dual-label immunofluorescence analysis revealed that enhanced α-synuclein was also detected within activated astrocytes of the substantia nigra following CBE exposure (FIG. 6), suggesting that similar mechanisms (e.g., abnormal protein accumulation and/or trafficking) could contribute to both astroglia as well as neurons. The presence of α-synuclein within astrocytes under these conditions could be relevant to both Gaucher disease and PD and/or PD-like diseases. It is possible that extracellular α-synuclein released from neurons is taken up into surrounding astroglia; indeed, such events have been hypothesized to contribute to astrocytic activation (Croisier and Graeber, 2006, Braak et al., 2007, Lee, 2008). Alternatively, it may be that upregulation is responsible for increased α-synuclein levels in astrocytes. α-synuclein has been detected in cultured human astrocytes, and its expression level is responsive to cytokine exposure (Tanji et al., 2001). Recently, Vitner and Futerman reported that astroglial cultures challenged with CBE have enhanced expression of α-synuclein mRNA, suggesting that increased transcription may contribute at least in part to enhanced α-synuclein levels in astrocytes (2008).

The increased α-synuclein and evidence of astrogliosis following a single dose, this indicates that even subtle changes in glucocerebrosidase activity may modify cellular α-synuclein metabolism and trigger a cascade of degenerative events.

Protein Levels of in α-Synuclein in Aged Mice:

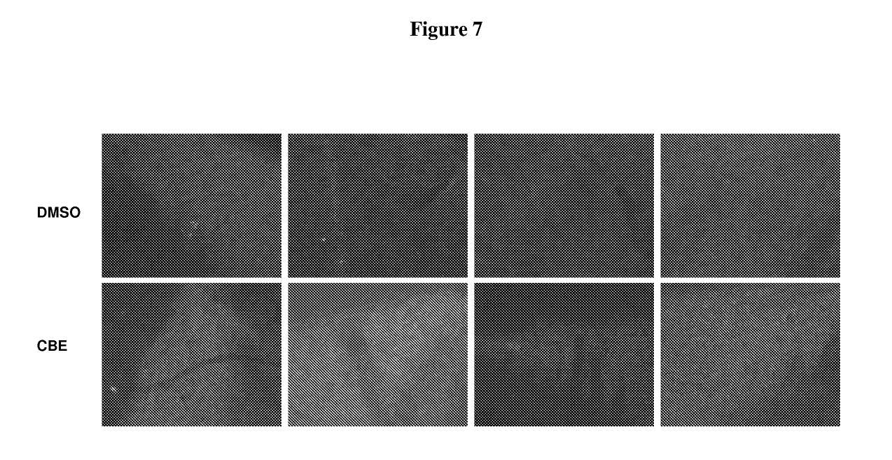

FIG. 7 shows increased α-synuclein expression within the substantia nigra of aged mice treated sub-chronically with CBE vs. DMSO. It is likely that sustained glucocerebrosidase inhibition promotes increased levels of the α-synuclein expression protein.

The increased proteins levels of α-synuclein and the lack of transcriptional change in α-synuclein could be due to decreased degradation. The ubiquitin-proteasome system may be compromised (see review, Hruska et al. 2006). However, additionally or alternatively altered lysosomal function could interfere with α-synuclein clearance in the animal. The alteration in distribution of α-synuclein with CBE suggests that the normal subcellular localization (and consequently normal function) of the protein may be disrupted in Gaucher disease.

Decreased lysosomal α-synuclein clearance and/or binding of the protein to accumulating glycolipids (Lee et al. 2004; Schlossmacher et al. 2005) could also contribute to the alteration of normal α-synuclein metabolism, trafficking and ultimately, function. In support of this possibility, data presented here show that the normal cellular distribution of α-synuclein is perturbed after GCase inhibition by CBE. After CBE exposure to mice, accumulation of the protein within neuronal cell bodies (FIGS. 3, 4) is seen, and further, that ventral mesencephalon levels of α-synuclein were increased in the particulate fraction (FIG. 4), suggesting an alteration in α-synuclein solubility and/or its trafficking. Under non-pathological conditions, α-synuclein co-localizes with lipid rafts that mediate its delivery to the synapse, but under conditions of altered lipid metabolism, this association is disrupted (Fortin et al., 2004). Consequently, redistribution of the protein to the cell body from neurites occurs, a scenario that could lead to the formation of abnormal and potentially toxic α-synuclein species (Fortin et al., 2004). In this setting, disruption of normal α-synuclein-lipid interactions, due to diminished GCase activity or other regulators of lipid metabolism, could represent a pathway that leads to cellular degeneration and/or cell demise.

Cellular Degeneration:

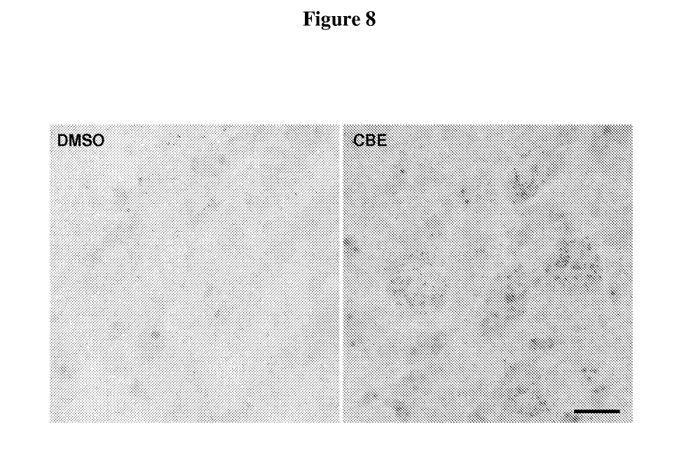

FIG. 8 shows accumulation of silver grains in nigral neurons from CBE- but not DMSO-treated mice. This indicates degenerating neurons within the substantia nigra of CBE-treated mice, demonstrating that glucocerebrosidase inhibition results in nigral cell death in animals.

### Example 4

### Alterations in α-Synuclein in Human Brains

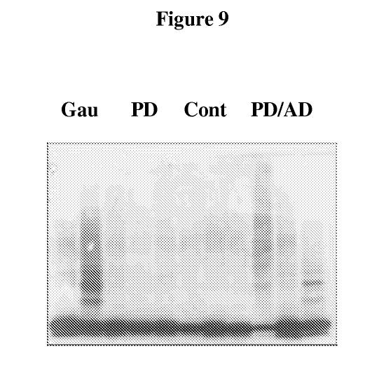

FIG. 9 shows α-synuclein alterations in the brains of patients with Parkinson's disease who carry a Gaucher mutation. Pictured is a Western blot analysis of α-synuclein of samples from a Gau+/− brain.

### Example 5

### Administration of a Pharmaceutical Composition of Formula 2 for the Treatment of Parkinson's Disease

A 63 year old male is diagnosed with Parkinson's disease. He is diagnosed upon undergoing a battery of motor testing. The patient is administered a pharmaceutical composition of the compound of Formula 2, wherein the administration is a single oral tablet, taken about 15 minutes after each of 3 meals a day. After continuation of the medication for a period of about 180 days, the patient's motor status is assessed.

### Example 6

### Administration of a Pharmaceutical Composition of an α-Synuclein Modulating Agent for the Treatment of Gaucher Disease

A 28 year old female is diagnosed with Gaucher disease. In addition to physiological measures such as mild osteoporosis, anemia, changes in spleen size (splenomegaly), and pigmentation alterations of the skin, she is genetically tested. Upon genetic testing it is determined that she carries a homozygous recessive point mutation, N370S, in the beta-glucosidase gene. She exhibits no outward neurological symptoms except for some occasional forgetfulness, which is not necessarily determined to be caused by the disease process. She enrolls in a double-blinded clinical trial where pharmaceutical compositions of 5 compounds of Table 1 are being tested for their capacity to reduce the symptoms of Gaucher disease, specifically by modulating α-synuclein. She is administered one of the compounds for a period of about 90 days, during which she discontinues her other Gaucher disease related medications. She is administered the drug 2 times per day for the period of 90 days, sublingually. At the end of the 90 days, several physiological variables are measured to measure her response on the clinical trial, including splenic measurements, assessment of her bone status, assessment of her anemic status, and assessment of skin pigmentation.

1. A method of treating a condition in a subject in need of treatment comprising administering an agent that alters lipid metabolism to the subject, wherein the condition is characterized by α-synuclein dysfunction.

2. The method of claim 1 wherein the condition is selected from the group consisting of: Parkinson's disease, Parkinson's disease with accompanying dementia, Lewy body dementia, Lewy body variant of Alzheimer's disease, Huntington's disease, Alzheimer's disease with Parkinsonism, and multiple system atrophy.

3. The method of claim 1 wherein α-synuclein dysfunction is further characterized by a dysfunction in α-synuclein fibrillation, ubiquitination, trafficking, subcellular compartmentalization, synaptic targeting, lysosomal storage, and lipid-interactions.

4. The method of claim 1 wherein the lipid metabolism is altered by decreasing ceramide levels.

5. The method of claim 4 wherein ceramide levels are decreased by MDR inhibitors.

6. The method of claim 1 wherein lipid metabolism is altered by decreasing a buildup of at least one glycosphingolipid.

7. The method of claim 1 wherein lipid metabolism is altered by altering glycosphingolipid metabolism.

8. The method of claim 6 or 7 wherein the glycosphingolipid is glucocerebroside.

9. The method of claim 1 wherein the agent that alters lipid metabolism is selected from the group consisting of: MDR inhibitors, glucocerebrosidases, and HMG-CoA reductase inhibitors.

10. The method of claim 9 wherein the agent is a HMG-CoA reductase inhibitor and the HMG-CoA reductase inhibitor is a statin.

11. The method of claim 9 wherein the agent is a MDR inhibitor and the MDR inhibitor is chosen from the imidazole derivatives and compounds of Formula 1a, 1b, or 2.

12. The method of claim 11 wherein the wherein a compound of Formula 2 has the following formula 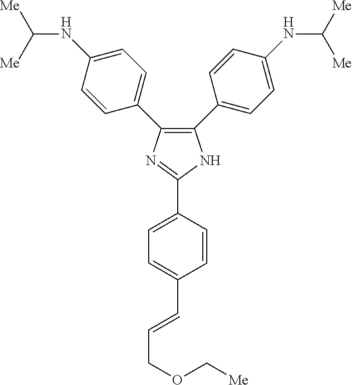 [SMILES: CCOC/C=C/c1ccc(-c2nc(-c3ccc(NC(C)C)cc3)c(-c3ccc(NC(C)C)cc3)[nH]2)cc1]
in the form of a free compound or as its pharmaceutically-acceptable pro-drug, metabolite, analogue, derivative, solvate or salt.

13. The method of claim 9 wherein the agent is an MDR inhibitor and the MDR inhibitor is chosen from the group consisting of: calcium channel blockers, calmodulin inhibitors, antibiotica, cardiovascular agents, noncytotoxic analogs of anthracyclines and vinca alkaloids, cyclosporine A, FK-506, and derivatives of cyclopeptides.

14. A method of treating a condition in a subject in need of treatment comprising administering an agent that corrects α-synuclein dysfunction to the subject, wherein the condition is characterized by altered lipid metabolism.

15. The method of claim 14 wherein the altered lipid metabolism is an accumulation of glucocerebroside.

16. The method of claim 14 wherein the condition is selected from the group consisting of: Gaucher disease, Fabry disease, lysosomal storage diseases, lipid storage diseases, glycoprotein storage diseases, mucolipidoses, gangliosidoses, leukodystrophies, mucopolysaccharidoses, Niemann-Pick disease, Tay Sachs diseases, Hunter syndrome, Hurler disease, Sandhoff's disease and cystic fibrosis.

17. The method of claim 14 wherein the agent that corrects α-synuclein dysfunction is selected from the group consisting of apomorphine, pyrogallol, 1,4-naphthoquinone, cisplatin, isoproterenol, pyrogallin, cianidanol, sulfasalazine, quinalizarin, benserazide, hexachlorophene, pyrvinium pamoate, dobutamine, methyl-dopa, curcumin, berberine chloride, daidzein, merbromin, norepinephrine, dopamine hydrochloride, carbidopa, ethylnorepinephrine hydrochloride, tannic acid, elaidyphosphocholine, hydroquinone, chlorophyllide Cu complex Na salt, methyldopa, isoproterenol hydrochloride, benserazide hydrochloride, dopamine, dobutamine hydrochloride, thyroid hormone, purpurin, sodium beta-nicotinamide adenine dinucleotide phosphate, lansoprazole, dyclonine hydrochloride, pramoxine hydrochloride, azobenzene, cefamandole sodium, cephaloridine, myricetin, 6,2′,3′-trihydroxyflavone, 5,7,3′,4′,5′-pentahydroxyflavone, 7,3′,4′,5′-tetrahydroxyflavone, (5,6,7,4′-tetrahydroxyflavone), baicalein, eriodictyol, 7,3′,4′-trihydroxyisoflavone, epigallocatechin gallate, quercetin, gossypetin (3,5,7,8,3′,4′-hexahydroxyflavone), 2′,3′-dihydroxyflavone, 3′,4′-dihydroxyflavone, 5,6-dihydroxy-7-methoxyflavone, baicalein-7-methyl ether, 1-dopa, DOPAC, homogentisic acid, 6-hydroxydopamine, epinephrine, 3,4-dihydroxycinnamic acid, 2,3-dihydroxynaphthalene, 3,4-dihydroxybenzoic acid, 3,4,5-trihydroxybenzoic acid, 1,2,3-trihydroxybenzoic acid, gallate (gallic acid), benzoquinone, catechol, rifampicin, rosmarinic acid, baicalin, tanshinones I and II, emodin, procyanidin B4, resveratrol, rutin, fisetin, luteolin, fustin, epicatechin gallate, catechin, alizarin, tannic acid, eriodyctol, carboplatin, purpurogallin-4-carboxylic acid, koparin, 2,3,4-trihydroxy-4′-ethexybenzophenone, baeomycesic acid, hamtoxylin, iriginol hexaaceatate, 4-acetoxyphenol, theaflavin monogallate, theaflavin digallate, stictic acid, purpurogallin, 2,5-dihydroxy-3,4-dimethoxy-4′-ethoxybenzophenone, promethazine hydrochloride, oxidopamine hydrochloride, pyrantel pamoate, elaidylphosphocholine, amphotericin B, gallic acid, fumarprotocetraric acid, theaflavin, haematoxylin pentaacetate, 4-methoxydalbergione, epigallocatechin-3-monogallate, rolitetracycline, 7,3′-dimethoxyflavone, liquiritigenin dimethyl ether, catechin pentaacetate, apigenin, 3,4-dedesmethyl-5-deshydroxy-3′-ethoxyscleroin, derivatives and analogs thereof.

18. The method of claim 7 wherein the glycosphingolipid is glucocerebroside.
